SEQUENCE LISTING (4 sequences)

SEQ ID NO: 1 - DNA, 20 bp, Artificial
  F2 forward primer sequence
aggcctgacatcaactccac

SEQ ID NO: 2 - DNA, 20 bp, Artificial
  F2 reverse primer sequence
ttggaacctcttgagcgagg

SEQ ID NO: 3 - DNA, 23 bp, Artificial
  GAPDH forward primer sequence
caaggtcatccatgacaactttg

SEQ ID NO: 4 - DNA, 20 bp, Artificial
  GAPDH reverse primer sequence
gggccatccacagtcttctg

(19) United States
(12) Patent Application Publication
(10) Pub. No.: US 2023/0340149 A1
(43) Pub. Date: Oct. 26, 2023

(54) METHODS OF TREATMENT OF INFLAMMATORY BOWEL DISEASES

(71) Applicants: INSERM (INSTITUT NATIONAL DE LA SANTÉ ET DE LA RECHERCHE MÉDICALE), Paris (FR); UNIVERSITÉ TOULOUSE III - PAUL SABATIER, Toulouse (FR); ECOLE NATIONALE VÉTÉRINAIRE DE TOULOUSE, Toulouse (FR); INSTITUT NATIONAL DE RECHERCHE POUR L'AGRICULTURE, L'ALIMENTATION ET L'ENVIRONNEMENT (INRAE), Paris (FR)

(72) Inventors: Nathalie VERGNOLLE, Toulouse (FR); Jean-Paul MOTTA, Toulouse (FR); Celine DERAISON, Toulouse (FR)

(21) Appl. No.: 18/044,216

(22) PCT Filed: Sep. 6, 2021
(86) PCT No.: PCT/EP2021/074438

§ 371 Date: Mar. 7, 2023

(30) Foreign Application Priority Data
Sep. 7, 2020 (EP) 20305988.6

(51) Int. Cl. C07K 16/36; A61K 38/57; A61P 1/04; C12N 15/113; C12N 15/115; A61K 35/741

(52) U.S. Cl. CPC C07K 16/36; A61K 38/57; A61P 1/04; C12N 15/1137; C12N 15/115; A61K 35/741; C07K 2317/76; C12N 2310/14; C12N 2310/11; C12N 2310/531

(57) ABSTRACT

Inventor's general objective was to investigate the potential role of mucosal thrombin in intestinal inflammation and its mechanisms of action. First, they evaluated whether there is an increased presence of active thrombin in Crohn's Disease (CD): both in patient tissues and in an animal models of IBD. Second, they investigated the effects on mucosal damage and tissue dysfunction resulting from the intracolonic administration of thrombin at a dose comparable to what was detected in the tissue of CD patients. Third, they demonstrated in IBD mouse model that pharmacological inhibition of mucosal thrombin activity is a new therapeutic approach to this debilitating condition, such inhibition, allows to significantly decrease all inflammatory parameters including the fecal bleeding score. Finally, inventors showed that human mucosa-associated commensal biofilms exposed to increasing concentrations of human thrombin exhibited increase in their virulent properties specifically, increased bacterial invasion into human epithelial cell line). So, the present invention relates to a method for preventing or treating inflammatory bowel diseases (IBD) such as Crohn's disease (CD) and ulcerative colitis (UC) by targeting locally the mucosal Thrombin.

### FIELD OF THE INVENTION

The present invention relates to a method for preventing or treating inflammatory bowel diseases (IBD) such as Crohn's disease (CD) and ulcerative colitis (UC) by targeting locally the mucosal Thrombin.

### BACKGROUND OF THE INVENTION

The pathogenesis of inflammatory Bowel Diseases (IBD) which include Crohn's disease (CD) and ulcerative colitis (UC) results from the complex interplay between genetic susceptibility, environmental input and immunological disorders¹. To date, therapeutic approaches for IBD have targeted predominantly the infiltration of inflammatory cells into the colonic tissue. However, it has been well established that leaky barrier and tissue dysfunctions also play roles in the pathology of IBD, complementing the immune system overactivation². Among the mucosal factors that could contribute to tissue malfunctions, serine proteases are of particular interest³. Indeed, increased serine protease activity has been associated with both CD and UC, when measured in tissue biopsies⁴⁻⁷ or in feces of IBD patients⁸, compared to healthy controls. In a recent study, inventors have used activity-based probes in order to identify the proteases that were the most actively released by IBD patient tissues. This study identified that thrombin was the most active protease associated with IBD patient tissues, and was 100 times more active in biopsy supernatants of Crohn's disease patients, compared to healthy controls⁵. This increased thrombin activity in the colon of CD patients can originate either from the general circulation, or from the intestinal epithelium, which we have identified as a source of active thrombin⁹.

Increased systemic thrombin generation was observed in IBD patient's blood, particularly in patients with high C-reactive protein levels¹¹. Coagulation cascade or platelet activation markers (including beta-thromboglobulin, D-dimer, or thrombin activatable fibrinolysis inhibitor) are associated with increased disease activity in Crohn's disease patients¹²⁻¹⁴. Acquired hypercoagulation, along with microthrombi formation in bowel capillaries of IBD patients have been reported^{15,16}. Further, treatments of IBD patients with the anticoagulant, heparin (an indirect thrombin inhibitor), has demonstrated some benefits¹⁷⁻¹⁹. This result was rather counter-intuitive considering the pro-coagulant role of thrombin and the fact that IBD patients experience mucosal bleeding. However, neither a proinflammatory role for thrombin in IBD nor its potential mechanism of action have yet been demonstrated.

### SUMMARY OF THE INVENTION

A first object of the invention relates to a direct thrombin inhibitor for use in the treatment of a patient affected with an inflammatory bowel disease (IBD) said direct thrombin inhibitor being administered locally in the Gut of the patient/subject to be treated.

In a particular embodiment, the inflammatory bowel disease (IBD) is selected from the list consisting of Ulcerative colitis (UC) and Crohn's disease (CD)

A second object of the invention relates to a recombinant food-grade bacterium comprising a gene selected from a gene coding for a member of the serpin family proteins or an active fraction of a member of the serpin family proteins (AntiThrombin, Heparin Cofactor II, Protein C inhibitor and protease Nexin 1) for use in the treatment of a patient affected with an inflammatory bowel disease (IBD) said recombinant food-grade bacterium being administered locally in the gut of the patient to be treated

### DETAILED DESCRIPTION OF THE INVENTION

### Therapeutic Methods and Uses

In the present invention, inventor's general objective was to investigate the potential role of mucosal thrombin in intestinal inflammation and its mechanisms of action. First they evaluated whether there is an increased presence of active thrombin in Crohn's Disease (CD): both in patient tissues and in an animal models of IBD. Second, they investigated the effects on mucosal damage and tissue dysfunction resulting from the intracolonic administration of thrombin in wild type and Protease-Activated Receptor (PAR)-1 or PAR4-null mice at a dose comparable to what was detected in the tissue of CD patients. Finally, they evaluated the effects of thrombin or PAR-1 and PAR-4 inhibition in a model of IBD and demonstrated that pharmacological inhibition of mucosal thrombin activity (and PAR-1 receptor activated by thrombin) is a new therapeutic approach to this debilitating condition. Furthermore the pharmacological inhibition of mucosal thrombin activity in IBD mouse model, allows to significantly decrease all inflammatory parameters including the fecal bleeding score. This result was rather counter-intuitive considering the pro-coagulant role of thrombin and the fact that IBD patients experience mucosal bleeding.

Finally, as demonstrated in example 2, inventors showed that human mucosa-associated commensal biofilms exposed to increasing concentrations of human thrombin exhibited increase in their virulent properties (specifically, increased bacterial adhesion to human epithelium and increased bacterial invasion into human epithelial cell line). Such increased virulence of human intestinal microbiota is indeed associated with IBD (King S J & McCole D F, 2019 Intestinal Res. 17(2):177-191).

Thus, the present invention provides methods and compositions (such as pharmaceutical compositions) for preventing or treating an inflammatory bowel disease (IBD).

In the context of the invention, the term “treatment or prevention” means reversing, alleviating, inhibiting the progress of, or preventing the disorder or condition to which such term applies, or one or more symptoms of such disorder or condition. In particular, the treatment of the disorder may consist in reducing the number of mucosal damage and tissue dysfunctions as observed in inflammatory bowel disease (IBD). Most preferably, such treatment leads to the complete depletion of the pathological features as observed in inflammatory bowel disease (IBD).

Preferably, the individual to be treated is a human or non-human mammal (such as a rodent a feline, a canine, or a primate) affected or likely to be affected with mucosal damage and tissue dysfunctions as observed in inflammatory bowel disease (IBD).

Preferably, the individual is a human.

Direct Thrombin Inhibitors

According to a first aspect, the present invention relates to a direct thrombin inhibitor (DTI) for use in the treatment of a patient affected with an inflammatory bowel disease (IBD), said direct thrombin inhibitor being administered locally in the Gut of the patient to be treated.

In the present invention, inventors demonstrated that thrombin expressed at mucosal surface of the gut is directly involved in the proinflammatory process in IBD, mainly via activation of PAR-1 receptor.

Accordingly, in order to be administered directly to the intestine (gut), the direct thrombin inhibitor is administered to the IBD patient orally (including buccal and sublingual administration), rectally or topically (intracolic administration),

When administered orally rectally or topically, the compound according to the invention can be in the form of a sustained release composition, or can be produced by a recombinant bacteria so as to deliver the inhibitor directly to the intestine.

In a particular embodiment, the inflammatory bowel disease (IBD) is selected from the list consisting of Ulcerative colitis (UC) and Crohn's disease (CD)

As used herein the term “thrombin” As used herein, “thrombin” denotes the activated enzyme (also known as fibrinogenase, thrombase, thrombofort, topical, thrombin-C, tropostasin, activated blood-coagulation factor II, blood-coagulation factor IIa, factor IIa, E thrombin, beta-thrombin, gamma-thrombin, meizothrombin), which results from the proteolytic cleavage of prothrombin (factor II).

Thrombin is a serine protease (EC 3.4.21.5), an enzyme that, in humans, is encoded by the F2 gene (Gene ID: 2147). Prothrombin (coagulation factor II) is proteolytically cleaved by prothrombinase complex (serine protein, Factor Xa, and the protein cofactor, Factor Va) to form thrombin in the clotting process. The molecular weight of prothrombin is approximately 72 kDa. The catalytic domain is released from prothrombin fragment 1.2 to create several active enzyme thrombin, meizothrombin at 50,000 Da, thrombin alpha at 32,000 Da, thrombin beta 28,000 Da, thrombin gamma 15,000 Da. (Chinnaraj M, et al. Sci Rep 8, 2945 (2018); Chen Z, et al. Proc Natl Acad Sci USA 107, 19278-19283 (2010); Matafonov A, et al. Blood 118, 437-445 (2011).)

The fully assembled prothrombinase complex (Factor Xa and Factor Va, phospholipid membranes, and Ca2+) catalyses the conversion of the zymogen prothrombin to the serine protease thrombin. Specifically, Factor Xa cleaves prothrombin in two locations, following Arg271 and Arg320 in human prothrombin. Because there are two cleavage events, prothrombin activation can proceed by two pathways. In one pathway, prothrombin is first cleaved at Arg271. This cleavage produces Fragment 1•2, comprising the first 271 residues, and the intermediate prethrombin 2, which is made up of residues 272-579. Fragment 1•2 is released as an activation peptide, and prethrombin 2 is cleaved at Arg320, yielding active thrombin. The two chains formed after the cleavage at Arg320, termed the A and B chains, are linked by a disulfide bond in active thrombin. In the alternate pathway for thrombin activation, prothrombin is first cleaved at Arg320, producing a catalytically active intermediate called meizothrombin. Meizothrombin contains fragment 1•2 A chain linked to the B chain by a disulfide bond. Subsequent cleavage of meizothrombin by Factor Xa at Arg271 gives Fragment 1•2 and active thrombin, consisting of the A and B chains linked by a disulfide bond. When thrombin is generated by Factor Xa alone, the first pathway predominates and prothrombin is first cleaved after Arg271, producing prethrombin 2, which is subsequently cleaved after Arg320. If Factor Xa acts as a component of the prothrombinase complex, however, the second pathway is favored, and prothrombin is first cleaved after Arg320, producing meizothrombin, which is cleaved after Arg271 to produce active thrombin. Thus, the formation of the prothrombinase complex alters the sequence of prothrombin bond cleavage.

Accordingly in a particular embodiment, the thrombin which can be targeted in the present invention are active or activated form of thrombin (Prothrombinase-mediated and autolytic-mediated degradation of prothrombin results in the formation of different forms of active thrombin):
Prothrombin (1-579 AA residues for Human form)
Prethrombin 2 (intermediate pathway 1: residues 272-579 of Prothrombin)
Meizothrombin (intermediate pathway 1: fragment 1•2 A chain (residues 1-320 of Prothrombin) linked to the B chain (residues 321-579 of Prothrombin) by a disulfide bond)
Active thrombin (or alpha thrombin) (formed by A chain (Light Chain: residues 272-320 of Prothrombin) and B chain (Heavy chain or catalytic domain 321-579) linked by a disulfide bond of Prothrombin).

Beta- and gamma thrombin which are obtained after proteolysis of alpha thrombin (and which are described as active thrombin (in Chinnaraj M, et al. Sci Rep 8, 2945 (2018); Chen Z, et al. Proc Natl Acad Sci USA 107, 19278-19283 (2010); Matafonov A, et al. Blood 118, 437-445 (2011)) with an enzymatic activity (ie able to cleave PAR4 and PAR-1 receptors).

A “Thrombin inhibitor” or “Thrombin antagonist” refers to a molecule (natural or synthetic) capable of neutralizing, blocking, inhibiting, abrogating, reducing or interfering with the biological activities of thrombin including, for example, reduction or blocking the interaction for instance between thrombin and PAR-1 (Protease-Activated Receptor-1). Thrombin inhibitors or antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Inhibitors or antagonists also include, antagonist variants of the protein, siRNA molecules directed to a protein, antisense molecules directed to a protein, aptamers, and ribozymes against a protein. For instance, the thrombin inhibitor or antagonist may be a molecule that binds to thrombin and neutralizes, blocks, inhibits, abrogates, reduces or interferes with the biological activity of thrombin (such as inducing local (mucosal) inflammation through activation of PAR-1 (protease-activated receptor-1) after thrombin cleavage).

Accordingly, a “Direct Thrombin Inhibitor” or “DTI”, is a thrombin inhibitor/antagonist which directly binds to thrombin (protein or nucleic sequence (DNA or mRNA)) and neutralizes, blocks, inhibits, abrogates, reduces or interferes with the biological activity of thrombin. Direct thrombin inhibitors (DTIs) are already a class of medication that act as anticoagulants (delaying fibrin blood clotting) by directly inhibiting the protease thrombin (factor IIa, which transforms fibrinogen into fibrin).

In the context of the present invention, the direct thrombin inhibitor (i) directly binds to thrombin (protein or nucleic sequence (DNA or mRNA)) and (ii) inhibits mucosal inflammation (through blocking thrombin-induced PAR-1 (Protease-Activated Receptor-1) activation a process which then mediate the local inflammation in the gut) and/or inhibits virulent properties of human mucosa-associated microbial biofilms (decreased bacterial adhesion to human epithelial cell).

More particularly, the direct thrombin inhibitor according to the invention is:
1) an inhibitor of thrombin activity (such as small organic molecule, antibody, aptamer, polypeptide) and/or
2) an inhibitor of thrombin gene expression (such as antisense oligonucleotide, nuclease, siRNA, . . . )

By “biological activity” of thrombin is meant in the context of the present invention, inducing mucosal (local) inflammation in the Gut (through the PAR-1 (Protease-Activated Receptor-1) cleavage regarding the pro-inflammation PAR-1 activation) and/or increasing virulent properties of human mucosa-associated microbial biofilms (increased bacterial adhesion to human epithelial cells).

Tests for determining the capacity of a compound to be a thrombin inhibitor are well known to the person skilled in the art. In a preferred embodiment, the antagonist/inhibitor specifically binds to thrombin (protein or nucleic sequence (DNA or mRNA)) in a sufficient manner to inhibit the biological activity of thrombin. Binding to thrombin and inhibition of the biological activity of thrombin may be determined by any competing assays well known in the art. For example, the assay may consist in determining the ability of the agent to be tested as a thrombin thrombin to bind to thrombin. The binding ability is reflected by the Kd measurement. The term “Kd”, as used herein, is intended to refer to the dissociation constant, which is obtained from the ratio of Kd to Ka (i.e. Kd/Ka) and is expressed as a molar concentration (M). Kd values for binding biomolecules can be determined using methods well established in the art. In specific embodiments, an antagonist/inhibitor that “specifically binds to thrombin” is intended to refer to an inhibitor that binds to human thrombin polypeptide with a Kd of 1 M or less, 100 nM or less, 10 nM or less, or 3 nM or less. Then a competitive assay may be settled to determine the ability of the agent to inhibit biological activity of thrombin. The functional assays based on local (mucosal) inflammation parameters may be envisaged such as evaluating the ability to inhibit gut inflammation processes (in a murine IBD model) such as assaying the mucosal macroscopic damage and/or colon thickness (see example 1 and FIG. 2) and/or fecal bleeding scoring (see example 1 FIG. 5). Additional assays regarding systemic inflammation will be assessed specifically, cytokine dosage in the blood and complete blood count See the method for cytokine bead array described in Danese S. el Gastroenterology 2019; 157:1007-1018, to assess either circulating or tissue cytokines/. Another functional assay based on local gut inflammation markers may also be used such as evaluating the ability to inhibit the increased cytokines involved in the inflammatory response (ie TNF alpha). For instance inhibition of TNF alpha (Tumor Necrosis Factor Alpha) activity can be assessed by detecting active TNF alpha with specific antibody (immunoblotting analysis), or by detecting the local TNF alpha expression.

Finally, assays investigating inhibition of thrombin-induced virulent properties of human mucosa-associated microbial biofilms (typically, adherence to and invasion into intestinal epithelium)) and bacterial detachment from biofilms could also be used (using a microtiter device to grow human biofilms, the bacterial dispersion will be assessed by plate counting and resazurin metabolic conversion assay (Motta et al. 2015 Inflamm Bowel Dis. 2015 May; 21(5):1006-17 and Motta et al. Inflamm Bowel Dis. 2018 Jun. 8; 24(7):1493-1502)). The skilled in the art can easily determine whether a thrombin antagonist neutralizes, blocks, inhibits, abrogates, reduces or interferes with a biological activity of thrombin. to check whether the thrombin antagonist/inhibitor binds to thrombin and/or is able to inhibit processes associated with local (mucosal) Inflammation in the gut (for instance, through inhibition of PAR-1 (Protease-Activated Receptor-1) activity), and/or is able to inhibit virulent properties of human mucosa-associated microbial biofilms (typically, adherence to and invasion into intestinal epithelium), in the same way than the initially characterized inhibitor of thrombin, binding assay and/or a PAR-1 (Protease-Activated Receptor-1) activity assay and/or bacterial detachment from biofilms may be performed with each antagonist.

Accordingly, the direct thrombin inhibitor may be a molecule that binds to thrombin selected from the group consisting of small organic molecules antibodies, aptamers, and polypeptides.

The skilled in the art can easily determine whether a thrombin inhibitor or antagonist neutralizes, blocks, inhibits, abrogates, reduces or interferes with a biological activity of thrombin: (i) binding to thrombin (protein or nucleic sequence (DNA or mRNA)) and/or (ii), inhibiting mucosal inflammation through blocking the PAR-1 (Protease-Activated Receptor-1) activity and/or iii) inhibiting virulent properties of human mucosa-associated microbial biofilms.

The terms “inflammatory bowel disease (IBD)” refer to a group of inflammatory diseases of the colon and small intestine. The major types of IBD are Crohn's disease (CD), ulcerative colitis, Celiac disease, and pouchitis. Crohn's disease affects the small intestine and large intestine, as well as the mouth, esophagus, stomach and the anus, whereas ulcerative colitis primarily affects the colon and the rectum (Xavier R J, et al (July 2007 Nature. 448 (7152): 427-434).

Examples of IBD according to the invention include Ulcerative colitis (UC) Crohn's disease (CD).

Preferably the inflammatory bowel disease (IBD) is Crohn's disease (CD).

CD and UC are chronic inflammatory diseases, and are not medically curable except for the use of surgery, although this may not eliminate extra-intestinal symptoms, and for CD, this does not preclude relapses. Accordingly there is a medical need to specifically treat IBD patient with new therapeutic approach.

Examples of Direct Thrombin inhibitors include but are not limited to any of the thrombin inhibitors described in “Direct Thrombin Inhibitors”. (2011) British Journal of Clinical Pharmacology. 72 (4): all of which are herein incorporated by reference.

Typically, a direct thrombin inhibitors according to the invention includes but is not limited to:
A) Inhibitor of thrombin activity such as:
   i. thrombin inhibitors small molecule such as: Argatroban, Ximelagatran and Dabigatran
   ii. Anti-thrombin neutralizing antibody
   iii. Polypeptide such as Hirudin Derivatives that are capable of inhibiting thrombin activity such as hirudin, lepirudin (Refludan®), desirudin and bivalirudin (Angiomax®),
   iv. Endogenous polypeptide such as serpins family that are capable of inhibiting thrombin activity such as AntiThrombin, Heparin Cofactor II, Protein C inhibitor and protease Nexin 1.
   v. Kunitz-type polypeptide isolated from bloodsuckers parasites which have direct and highly specific thrombin inhibitors activity such as Sculptin Ornithodorin, and Savignin
   vi. Polysaccharide such as Dextran and derivatives that are capable of inhibiting thrombin activity
B) Inhibitor of thrombin gene expression selected from the list consisting of antisense oligonucleotide, nuclease, siRNA, shRNA or ribozyme nucleic acid sequence.

### A) Inhibitor of Thrombin Activity

Small Organic Molecule

In one embodiment, the thrombin antagonist is a small organic molecule. As used herein, the term “small organic molecule” refers to a molecule of size comparable to those organic molecules generally used in pharmaceuticals. The term excludes biological macromolecules (e.g.; proteins, nucleic acids, etc.); preferred small organic molecules range in size up to 2000 Da, and most preferably up to about 1000 Da.

Small molecular direct thrombin inhibitors (smDTIs) are non-peptide small molecules that specifically and reversibly inhibit both free and clot-bound thrombin by binding to the active site of the thrombin molecule. They prevent VTE in patients undergoing hip- and knee replacement surgery (Lee, C J.; et al (2011). “Direct Thrombin Inhibitors”. British Journal of Clinical Pharmacology. 72 (4): 581-592). The advantages of this type of DTIs are that they do not need monitoring, have a wide therapeutic index and the possibility of oral administration route. They are theoretically more convenient than both vitamin K antagonist and LMWH. Researches will, however, have to show the indication of the use and their safety (Squizzato, A; et al (2009). “New direct thrombin inhibitors”. Intern Emerg Med. 4 (6): 479-484).

Several Small molecular direct are disclosed below.

In a particular embodiment, the thrombin antagonist according to the invention is a small organic molecule such as:

i. Argatroban (PubChem Compound Identification: 440542)

Argatroban is a small univalent DTI formed from P1 residue from arginine. It binds to the active site on thrombin. (Lee, C J. (2011). “Direct Thrombin Inhibitors”. British Journal of Clinical Pharmacology. 72 (4): 581-592). The X-ray crystal structure shows that the piperidine ring binds in the S2 pocket and the guanidine group binds with hydrogen bonds with Asp189 into the S1 pocket. It's given as an intravenous bolus because the highly basic guanidine with pKa 13 prevents it to be absorbed from the gastrointestinal tract (Kikelj, Danijel. (2004). “Peptidomimetic Thrombin Inhibitors”. Pathophysiology of Haemostasis and Thrombosis. 33 (5-6): 487-491). The plasma half-life is approximately 45 minutes. As argatroban is metabolized via hepatic pathway and is mainly excreted through the biliary system, dose adjustments are necessary in patients with hepatic impairment but not renal damage. Argatroban has been approved in the USA since 2000 for the treatment of thrombosis in patients with HIT and 2002 for anticoagulation in patients with a history of HIT or are at risk of HIT undergoing percutaneous coronary interventions (PCI). (Lee, C J. (2011) and Kikelj, Danijel. (2004). It was first introduced in Japan in 1990 for treatment of peripheral vascular disorders (Kikelj, Danijel. (2004).

Argatroban has the following structure (where it binds to the S1 and S2 pockets):

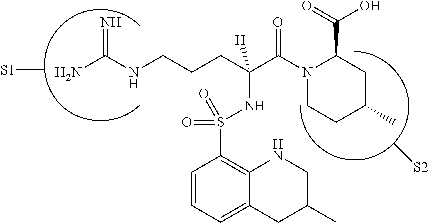

ii. Ximelagatran (PubChem Compound Identification: 9574101)

The publication of the NAPAP-fIIa crystal structure triggered many researches on thrombin inhibitors. NAPAP is an active site thrombin inhibitor. It fills the S3 and S2 pockets with its naphthalene and piperidine groups. AstraZeneca used the information to develop melagatran. The compound was poorly orally available, but after renovation they got a double prodrug which was the first oral DTI in clinical trials, ximelagatran (Nar, Herbert (2012). “The role of structural information in the discovery of direct thrombin and factor Xa inhibitors”. Trends in Pharmacological Sciences. 33 (5): 279-288.) Ximelagatran was on the European market for approximately 20 months when it was suspended. Studies showed that treatment for over 35 days was linked with the risk of hepatic toxicity (Squizzato, A et al (2009). “New direct thrombin inhibitors”. Intern Emerg Med. 4 (6): 479-484.) It was never approved by the FDA.

Ximelagatran (double prodrug turns into the active form melagatran in vivo) has the following structure:

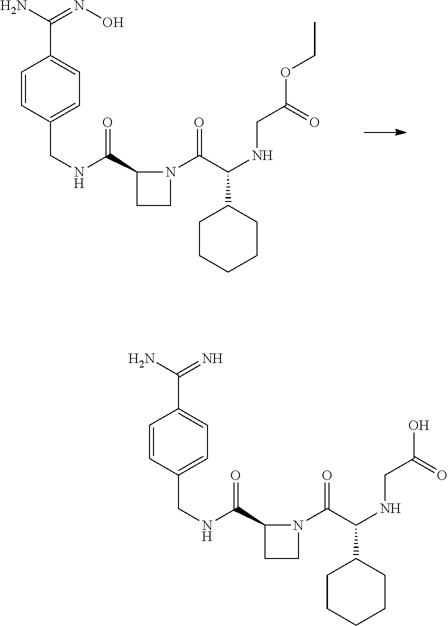 [SMILES: CCOC(=O)CN[C@@H](C(=O)N1CC[C@H]1C(=O)NCc1ccc(/C(N)=N\O)cc1)C1CCCCC1 ; N=C(N)c1ccc(CNC(=O)[C@@H]2CCN2C(=O)[C@H](NCC(=O)O)C2CCCCC2)cc1]

iii. Dabigatran ((PubChem Compound Identification: 135565674)

Researchers at Boehringer Ingelheim used the publicized information about the NAPAP-fIIa crystal structure, starting with the NAPAP structure that led to the discovery of dabigatran (Nar, H. (2012). Trends in Pharmacological Sciences. 33 (5): 279-288) which is a very polar compound and therefore not orally active. By masking the amidinium moiety as a carbamate-ester and turning the carboxylate into an ester they were able to make a prodrug called dabigatran etexilate, (Hauel, N.; et al (2002). Journal of Medicinal Chemistry. 45 (9): 1757-1766) a highly lipophilic, gastrointestinally absorbed and orally bioavailable double prodrug such as ximelagatran, with the plasma half-life of approximately 12 hours (Nar, H. (2012). Trends in Pharmacological Sciences. 33 (5): 279-288). Dabigatran etexilate is rapidly absorbed, it lacks interaction with cytochrome P450 enzymes and with other food and drugs, there is no need for routine monitoring and it has a broad therapeutic index and a fixed-dose administration, which is excellent safety compared with warfarin (O'Brien, P. et al (2012). “Direct Thrombin Inhibitors”. Journal of Cardiovascular Pharmacology and Therapeutics. 17 (1): 5-11). Unlike ximelagatran, a long-term treatment of dabigatran etexilate has not been linked with hepatic toxicity, seeing as how the drug is predominantly eliminated (>80%) by the kidneys. Dabigatran etexilate was approved in Canada and Europe in 2008 for the prevention of VTE in patients undergoing hip- and knee surgery. In October 2010 the US FDA approved dabigatran etexilate for the prevention of stroke in patients with atrial fibrillation (AF) (Thethi, I. et al (2012). “Oral Factor Xa and Direct Thrombin Inhibitors”. Journal of Burn Care & Research. 33 (4): 453-461). Many pharmaceutical companies have attempted to develop orally bioavailable DTI drugs but dabigatran etexilate is the only one to reach the market (Nar, Herbert (2012)).

Dabigatran (double prodrug Dabigatran etexilate turns into the active form Dabigatran in vivo) has the following structure (at left side BIBR1048; at the right side BIBR953):

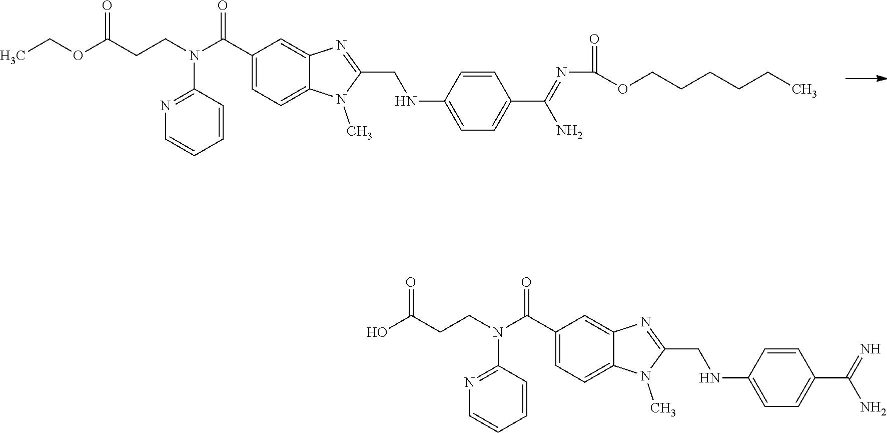 [SMILES: CCCCCCOC(=O)/N=C(\N)c1ccc(NCc2nc3cc(C(=O)N(CCC(=O)OCC)c4ccccn4)ccc3n2C)cc1 ; Cn1c(CNc2ccc(C(=N)N)cc2)nc2cc(C(=O)N(CCC(=O)O)c3ccccn3)ccc21]

If delivered via intrarectal bolus (direct administration), the direct thrombin inhibitor will have to be delivered in its active moiety Dabigatran BIBR953 and not in its oral formulation Dabigatran etexilate (BIBR1048).

Antibody

In another embodiment, the thrombin antagonist is an antibody (the term including antibody fragment or portion) that can block directly or indirectly the interaction of thrombin with PAR-1 (Protease-Activated Receptor-1).

In preferred embodiment, the thrombin antagonist may consist in an antibody directed against the thrombin, in such a way that said antibody impairs the binding of a thrombin to PAR-1 (Protease-Activated Receptor-1) and able of neutralizing, blocking, inhibiting, abrogating, reducing or interfering with the biological activities of thrombin (“neutralizing antibody”).

Then, for this invention, neutralizing antibody of thrombin are selected as above described for their capacity to (i) bind to thrombin (protein) and/or (ii) inhibiting mucosal inflammation through blocking the PAR-1 (Protease-Activated Receptor-1) activity and/or (iii) inhibiting virulent properties of human mucosa-associated microbial biofilms (typically, adherence to and invasion into intestinal epithelium).

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of thrombin. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant thrombin may be provided by expression with recombinant cell lines or bacteria. Recombinant form of thrombin may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as it is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3 A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

The skilled artisan can use routine technologies to use the antigen-binding sequences of these antibodies (e.g., the CDRs) and generate humanized antibodies for treatment of inflammatory bowel disease (IBD) (such as Crohn's disease (CD) as disclosed herein.

Several monoclonal antibodies to thrombin have been characterized and shown to inhibit thrombin activity (see Dawes J, et al. Thromb Res. 1984 Dec. 1; 36(5):397-409).

The skilled artisan can use routine technologies to use the antigen-binding sequences of these antibodies (e.g., the CDRs) and generate humanized antibodies for treatment of inflammatory bowel disease (IBD) (such as Crohn's disease (CD) as disclosed herein.

Aptamer

In another embodiment, the thrombin antagonist is an aptamer directed against thrombin. aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of thrombin are selected as above described for their capacity to (i) bind to thrombin and/or (ii) and inhibiting mucosal inflammation through blocking the PAR-1 (Protease-Activated Receptor-1) activity, and/or iii) inhibiting mucosa-associated microbiota pathogenicity (typically, adherence to intestinal epithelium).

Examples of neutralizing RNA aptamers of thrombin that can be used according to the invention are disclosed in the review Deng B, et al “Aptamer binding assays for proteins: The thrombin example—A review” Analytica Chimica Acta Volume 837, 21 Jul. 2014, p. 1-15.

Polypeptide

In another embodiment, the thrombin antagonist can be a polypeptide

As used herein, the term “thrombin polypeptide antagonist” refers to a polypeptide that specifically binds to Thrombin and be capable of inhibiting thrombin biological activity

In a specific embodiments the thrombin polypeptide antagonist is as thrombin mutant or enzyme (protease or peptidase)

In preferred embodiment, the thrombin polypeptide antagonist may consist in a polypeptide directed against the thrombin protein, and able of neutralizing, blocking, inhibiting, abrogating, reducing or interfering with the biological activities of thrombin (“neutralizing polypeptide”).

Then, for this invention, neutralizing polypeptide of thrombin are selected as above described for their capacity to (i) bind to thrombin (protein) and/or (ii) and inhibiting mucosal inflammation through blocking the PAR-1 (Protease-Activated Receptor-1) activity and/or inhibiting virulent properties of human mucosa-associated microbial biofilms (typically, adherence to and invasion into intestinal epithelium).

Examples of thrombin polypeptide antagonist that can be used according to the invention include:

1) Hirudin and Derivatives

Hirudin derivatives are all bivalent DTIs, they block both the active site and exosite 1 in an irreversible 1:1 stoichiometric complex. (O'Brien, P. et al (2012). “Direct Thrombin Inhibitors”. Journal of Cardiovascular Pharmacology and Therapeutics. 17 (1): 5-11) The active site is the binding site for the globular amino-terminal domain and exosite 1 is the binding site for the acidic carboxy-terminal domain of hirudin (Weitz, Jeffery I.; Crowther, Mark (2002). “Direct Thrombin Inhibitors”. Thrombosis Research. 106 (3): 275-284).

a) Hirudin

Native hirudin, a 65-amino-acid polypeptide, is produced in the parapharyngeal glands of medicinal leeches (Greinacher, A et al (2008). “The direct thrombin inhibitor hirudin”. Thrombosis and Haemostasis. 99 (5): 819-829). Hirudins today are produced by recombinant biotechnology using yeast. These recombinant hirudins lack a sulfate group at Tyr-63 and are therefore called desulfatohirudins. They have a 10-fold lower binding affinity to thrombin compared to native hirudin, but remain a highly specific inhibitor of thrombin and have an inhibition constant for thrombin in the picomolar range. (O'Brien, P. et al (2012) and Greinacher, A et al (2008)). Renal clearance and degradation account for the most part for the systemic clearance of desulfatohirudins and there is accumulation of the drug in patients with chronic kidney disease. These drugs should not be used in patients with impaired renal function, since there is no specific antidote available to reverse the effects (O'Brien, P. et al (2012)). Hirudins are given parenterally, usually by intravenous injection. 80% of hirudin is distributed in the extravascular compartment and only 20% is found in the plasma. The most common desulfatohirudins today are lepirudin and desirudin. (Greinacher, A et al (2008)).

In a meta-analysis of 11 randomized trials involving hirudin and other DTIs versus heparin in the treatment of acute coronary syndrome (ACS) it was found that hirudin has a significantly higher incidence of bleeding compared with heparin. Hirudin is therefore not recommended for treatment of ACS and currently it has no clinical indications (O'Brien, P. et al (2012). “Direct Thrombin Inhibitors”. Journal of Cardiovascular Pharmacology and Therapeutics. 17 (1): 5-11)

b) Lepirudin

Lepirudin was previously approved for the treatment of heparin-induced thrombocytopenia (HIT) in the USA, Canada, Europe and Australia. Lepirudin was discontinued for human use in 2012 by Bayer pharmaceutical. HIT is a very serious adverse event related to heparin's use and occurs with both unfractionated heparin and LMWH, although to a lesser extent with the latter. It is an immune-mediated, prothrombotic complication which results from a platelet-activating immune response triggered by the interaction of heparin with platelet factor 4 (PF4) (Sakr, Y. (2011). “Heparin-induced thrombocytopenia in the ICU: an overview”. Critical Care. 15 (2): 211). The PF4-heparin complex can activate platelets and may cause venous and arterial thrombosis (Di Nisio, M et al. (2005). “Direct Thrombin Inhibitors”. New England Journal of Medicine. 353 (10): 1028-1040). When lepirudin binds to thrombin it hinders its prothrombic activity (Sakr, Y. (2011). Three prospective studies, called the Heparin-Associated-Thrombocytopenia (HAT) 1,2, and 3, were performed that compared lepirudin with historical controls in the treatment of HIT. All three studies showed that the risk of new thrombosis was decreased with the use of lepirudin, but the risk for major bleeding was increased (Greinacher, A. et al (2008). “Thrombosis and Haemostasis. 99 (5): 819-829). The higher incidence of major bleeding is thought to be the result of an approved dosing regimen that was too high, consequently the recommended dose was halved from the initial dose (O'Brien, P. et al (2012).

c) Desirudin

Desirudin is approved for treatment of venous thromboembolism (VTE) in Europe and multiple phase III trials are presently ongoing in the USA (O'Brien, P. et al (2012).) Two studies comparing desirudin with enoxaparin (a LMWH) or unfractionated heparin have been performed. In both studies desirudin was considered to be superior in preventing VTE. Desirudin also reduced the rate of proximal deep vein thrombosis. Bleeding rates were similar with desirudin and heparin ((O'Brien, P. et al (2012) and Di Nisio, M. et al (2005).

d) Bivalirudin

Bivalirudin, a 20 amino acid polypeptide, is a synthetic analog of hirudin. Like the hirudins it is also a bivalent DTI. It has an amino-terminal D-Phe-Pro-Arg-Pro domain that is linked via four Gly residues to a dodecapeptide analog of the carboxy-terminal of hirudin. The amino-terminal domain binds to the active site and the carboxy-terminal domain binds to exosite 1 on thrombin. Different from the hirudins, once bound thrombin cleaves the Arg-Pro bond at the amino-terminal of bivalirudin and as a result restores the functions to the active site of the enzyme. Even though the carboxy-terminal domain of bivalirudin is still bound to exosite 1 on thrombin, the affinity of the bond is decreased after the amino-terminal is released. This allows substrates to substrates to compete with cleaved bivalirudin for access to exosite 1 on thrombin (Weitz, J I.; et al (2002). “Direct Thrombin Inhibitors”. Thrombosis Research. 106 (3): 275-284). The use of bivalirudin has mostly been studied in the setting of acute coronary syndrome. A few studies indicate that bivalirudin is non-inferior compared to heparin and that bivalirudin is associated with a lower rate of bleeding.[4] Unlike the hirudins, bivalirudin is only partially (about 20%) excreted by the kidneys, other sites such as hepatic metabolism and proteolysis also contribute to its metabolism, making it safer to use in patients with renal impairment; however, dose adjustments are needed in severe renal impairment (Di Nisio, M. et al (2005) and Sakr, Yasser (2011).

Examples of thrombin polypeptide antagonist that can be used according to the invention also include

2) Endogenous Serpins Family

As used herein, the term “Serpin family” for SERine Protease INhibitors denotes a family of serine proteinase inhibitors which are similar in amino acid sequence and mechanism of inhibition, but differ in their specificity toward proteolytic enzymes. This family includes alpha 1-antitrypsin (A1-Pi), angiotensinogen, ovalbumin, antiplasmin, alpha 1-antichymotrypsin, thyroxine-binding protein, complement 1 inactivators, antithrombin III, heparin cofactor II, plasminogen inactivators, gene Y protein, placental plasminogen activator inhibitor, and barley Z protein. This family does not include the WAP-type four-disulfide core (WFDC) domain family, which include secretory leukocyte proteinase inhibitor (SLPI) and Elafin (aliases: elastase-specific inhibitor, skin-derived antileukoproteinase, protease-inhibitor 3, PI3) [Thierry Moreau et al., 2008]. Some members of the serpin family may be substrates rather than inhibitors of serine endopeptidases, and some serpins occur in plants where their function is not known.

Examples of serpins family polypeptide inhibitors of Thrombin: include but are not limited to any of the serpins family polypeptide described in Huntington J A. Thrombin inhibition by the serpins. J Thromb Haemost 2013; 11 (Suppl. 1): 254-64

In a particular embodiment of the invention, serpins family polypeptide that are capable of directly inhibiting thrombin activity is selected from the group consisting of AntiThrombin, Heparin Cofactor II, Protein C inhibitor and protease Nexin 1.
a) AntiThrombin,
   Antithrombin also termed Antithrombin III (AT III), serpin family C member 1 (SERPINC1) is a protein encoded in human by SERPINC1 gene (Gene ID 462). Antithrombin III is a plasma protease inhibitor and a member of the serpin superfamily. This protein inhibits thrombin as well as other activated serine proteases of the coagulation system, and it regulates the blood coagulation cascade. The protein includes two functional domains: the heparin binding-domain at the N-terminus of the mature protein, and the reactive site domain at the C-terminus. The inhibitory activity is enhanced by the presence of heparin. Numerous mutations have been identified for this gene, many of which are known to cause antithrombin-III deficiency which constitutes a strong risk factor for thrombosis.
b) Heparin Cofactor II
   Heparin cofactor II (HCII) also termed serpin family D member 1 (SERPIND1) is a protein encoded in human by the SERPIND1 gene (Gene ID: 3053). Heparin cofactor II is plasma serine protease that functions as a thrombin and chymotrypsin inhibitor. The protein is activated by heparin, dermatan sulfate, and glycosaminoglycans. Allelic variations in this gene are associated with heparin cofactor II deficiency.
c) Protein C inhibitor
   Protein C inhibitor ((PCI) also termed serpin family A member 5 (SERPINA5) PAI-3, PROC1, PCI is a protein encoded in human by the SERPINA5 gene (Gene ID: 5104). Protein C inhibitor is a serine protease inhibitor (serpin) that inhibits the activity of protein C (an anticoagulant). Protein C inhibitor is activated by heparin against thrombin (Huntington J A. J Thromb Haemost 2013; 11 (Suppl. 1): 254-64). Protein C inhibitor inhibits several serine proteases, not only protein C but also thrombin and various plasminogen activators and kallikreins, and it thus plays diverse roles in hemostasis and thrombosis in multiple organs.
d) Protease Nexin 1
   Protease Nexin 1 (PN-1) also termed serpin family E member 2 (SERPINE2), glia-derived nexin (GDN) is a protein encoded in human by the SERPINE2 gene (Gene ID: 5270). PN-1 inhibits various serine proteases in particular Thrombin, urokinase, plasmin and trypsin.

3) Kunitz-Type Polypeptide from Bloodsuckers Parasites

The Kunitz-type polypeptide from bloodsuckers parasites have been recently described as direct (and highly specific) thrombin inhibitors. Those polypeptide have interesting feature: 1/low if any glycosylation sites 2/can be produced by Prokaryote such as E. coli.

In a particular embodiment of the invention, Kunitz-type family polypeptide from bloodsucker parasites that are capable of directly inhibiting thrombin activity is selected from the group consisting of Sculptin Ornithodorin, and Savignin

a) Sculptin

Sculptin isolated from Amblyomma sculptum is a polypeptide of 168 residues having four similar repeats and evolutionary diverged from hirudin Sculptin is a competitive, specific and reversible inhibitor of thrombin with a Ki of 18.3±1.9 pM (k on 4.04±0.03×107 M-1 s-1 and k off 0.65±0.04×10-3 s-1). A single domain of sculptin alone retains ˜45% of inhibitory activity, which could bind thrombin in a bivalent fashion. The formation of a small turn/helical-like structure by active site binding residues of sculptin might have made it a more potent thrombin inhibitor. In addition, sculptin prolongs global coagulation parameters. (IQBAL A. Sci Rep. 2017; 7: 1431.)

b) Ornithodorin,

Ornithodorin, isolated from the blood sucking soft tick Ornithodoros moubata, is a potent (Ki=10(−12) M) and highly selective thrombin inhibitor Internal sequence homology indicates a two domain protein. Each domain resembles the Kunitz inhibitor basic pancreatic trypsin inhibitor (BPTI) and also the tick anticoagulant peptide (TAP) isolated from the same organism. (Van de Locht A yet al. EMBO J. 1996 Nov. 15; 15(22): 6011-6017).

c) Savignin,

Savignin isolated from the tick Ornithodoros savignyi is also a potent thrombin inhibitor. Savignin protein sequence shows high identity (63%) with ornithodorin The N-terminal amino acid residues of savignin bind inside the active site cleft, while the C-terminal domain of savignin has a net negative electrostatic potential and interacts with the basic fibrinogen recognition exosite of thrombin through hydrogen bonds and hydrophobic interactions. Savignin is a competitive, slow, tight-binding inhibitor that requires thrombin's fibrinogen-binding exo-site for optimal inhibition. Mans B J. Insect Biochem Mol Biol. 2002 July; 32(7):821-8).

Polysaccharide

In another embodiment, the thrombin antagonist can be a polysaccharide

As used herein, the term “thrombin polysaccharide antagonist” refers to a polysaccharide that specifically binds to Thrombin and be capable of inhibiting thrombin biological activity

In a preferred embodiments the thrombin polysaccharide antagonist is dextran sulphates and derivatives

In preferred embodiment, the thrombin polysaccharide antagonist may consist in a polysaccharide directed against the thrombin protein, and able of neutralizing, blocking, inhibiting, abrogating, reducing or interfering with the biological activities of thrombin (“neutralizing polysaccharide”).

Then, for this invention, neutralizing polysaccharide of thrombin are selected as above described for their capacity to (i) bind to thrombin (protein) and/or (ii) and inhibiting mucosal inflammation through blocking the PAR-1 (Protease-Activated Receptor-1) activity and/or inhibiting virulent properties of human mucosa-associated microbial biofilms (typically, adherence to and invasion into intestinal epithelium).

Examples of thrombin polysaccharide antagonist that can be used according to the invention include:

Dextran and Derivatives

Dextran is a complex branched glucan (polysaccharide derived from the condensation of glucose). IUPAC (International Union of Pure and Applied Chemistry) defines dextrans as “Branched poly-α-d-glucosides of microbial origin having glycosidic bonds predominantly C-1→C-6” (IUPAC. Compendium of Chemical Terminology, 2nd ed. (the “Gold Book”). Compiled by A. D. McNaught and A. Wilkinson. Blackwell Scientific Publications, Oxford (1997). Dextran chains are of varying lengths (from 3 to 2000 kilodaltons). The polymer main chain consists of α-1,6 glycosidic linkages between glucose monomers, with branches from α-1,3 linkages.

Dextran sulphate is a polysaccharide with a high molecular weight and a high sulphur content inhibit thrombin activity directly, but dextran sulphates do not activate antithrombin III. These characteristics of dextran sulphates differ from those of heparin (see Suzuki K et al J Clin Pathol. 1979 May; 32(5): 439-444).

Dextran Derivatives

Several dextran derivatives have been developed with anticoagulant activities such as, short chain-length dextran sulphates (Ricketts C R Brit. J. Pharmacol. (1954), 9, 224), and carboxymethyl dextran benzylamide sulfonate/sulfates (CMDBS) which anticoagulant activity was due both to direct thrombin inhibition and to catalysis of thrombin inhibition by heparin cofactor II (HCII) (E. de Raucourt at al. Journal of Biomedical Materials Research Volume41, Issue1, July 1998. 49-57)

B) Inhibitor of Thrombin Gene Expression

In still another embodiment, the thrombin antagonist is an inhibitor of thrombin gene expression. An “inhibitor of expression” refers to a natural or synthetic compound that has a biological effect to inhibit the expression of a gene. Therefore, an “inhibitor of thrombin gene expression” denotes a natural or synthetic compound that has a biological effect to inhibit the expression of thrombin gene (or the gene transcript: RNA).

In a preferred embodiment of the invention, said inhibitor of thrombin gene expression is antisense oligonucleotide, nuclease, siRNA, shRNA or ribozyme nucleic acid sequence.

Inhibitors of thrombin gene expression for use in the present invention may be based on antisense oligonucleotide constructs. Antisense oligonucleotides, including antisense RNA molecules and antisense DNA molecules, would act to directly block the translation of thrombin mRNA by binding thereto and thus preventing protein translation or increasing mRNA degradation, thus decreasing the level of thrombin, and thus activity, in a cell. For example, antisense oligonucleotides of at least about 15 bases and complementary to unique regions of the mRNA transcript sequence encoding thrombin can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of thrombin gene expression for use in the present invention. Thrombin gene expression can be reduced by using small double stranded RNA (dsRNA), or a vector or construct causing the production of a small double stranded RNA, such that thrombin gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Example of commercial siRNAs against Thrombin are available.

Inhibitors of thrombin gene expression for use in the present invention may be based nuclease therapy (like Talen or Crispr).

The term “nuclease” or “endonuclease” means synthetic nucleases consisting of a DNA binding site, a linker, and a cleavage module derived from a restriction endonuclease which are used for gene targeting efforts. The synthetic nucleases according to the invention exhibit increased preference and specificity to bipartite or tripartite DNA target sites comprising DNA binding (i.e. TALEN or CRISPR recognition site(s)) and restriction endonuclease target site while cleaving at off-target sites comprising only the restriction endonuclease target site is prevented.

The guide RNA (gRNA) sequences direct the nuclease (i.e. Cas9 protein) to induce a site-specific double strand break (DSB) in the genomic DNA in the target sequence.

Restriction endonucleases (also called restriction enzymes) as referred to herein in accordance with the present invention are capable of recognizing and cleaving a DNA molecule at a specific DNA cleavage site between predefined nucleotides. In contrast, some endonucleases such as for example Fokl comprise a cleavage domain that cleaves the DNA unspecifically at a certain position regardless of the nucleotides present at this position. Therefore, preferably the specific DNA cleavage site and the DNA recognition site of the restriction endonuclease are identical. Moreover, also preferably the cleavage domain of the chimeric nuclease is derived from a restriction endonuclease with reduced DNA binding and/or reduced catalytic activity when compared to the wildtype restriction endonuclease.

According to the knowledge that restriction endonucleases, particularly type II restriction endonucleases, bind as a homodimer to DNA regularly, the chimeric nucleases as referred to herein may be related to homodimerization of two restriction endonuclease subunits. Preferably, in accordance with the present invention the cleavage modules referred to herein have a reduced capability of forming homodimers in the absence of the DNA recognition site, thereby preventing unspecific DNA binding. Therefore, a functional homodimer is only formed upon recruitment of chimeric nucleases monomers to the specific DNA recognition sites. Preferably, the restriction endonuclease from which the cleavage module of the chimeric nuclease is derived is a type llP restriction endonuclease. The preferably palindromic DNA recognition sites of these restriction endonucleases consist of at least four or up to eight contiguous nucleotides. Preferably, the type llP restriction endonucleases cleave the DNA within the recognition site which occurs rather frequently in the genome, or immediately adjacent thereto, and have no or a reduced star activity. The type llP restriction endonucleases as referred to herein are preferably selected from the group consisting of: Pvull, EcoRV, BamHl, Bcnl, BfaSORF1835P, BfiI, Bgll, Bglll, BpuJl, Bse6341, BsoB1, BspD6I, BstYl, Cfr101, Ecl18kl, EcoO109l, EcoRl, EcoRll, EcoRV, EcoR124l, EcoR124ll, HinP11, Hincll, Hindlll, Hpy99l, Hpy188l, Mspl, Munl, Mval, Nael, NgoMIV, Notl, OkrAl, Pabl, Pacl, PspGl, Sau3Al, Sdal, Sfil, SgrAl, Thal, VvuYORF266P, Ddel, Eco57l, Haelll, Hhall, Hindll, and Ndel.

Example of commercial gRNAs against thrombin include, but are not limited to:

F2 CRISPR guide RNA, coagulation factor II, thrombin CRISPR guide RNA[human] from GenScrip (disclosed in Sanjana N. E et al. Improved vectors and genome-wide libraries for CRISPR screening. Nat Methods. 2014 August; 11(8):783-4); Thrombin R CRISPR/Cas9 KO Plasmid (m): (sc-420264) from Santa Cruz Biotechnology, Thrombin R CRISPR/Cas9 KO Plasmid (h2): (sc-400556-KO-2) from Santa Cruz Biotechnology.

Other nuclease for use in the present invention are disclosed in WO 2010/079430, WO2011072246, WO2013045480, Mussolino C, et al (Curr Opin Biotechnol. 2012 October; 23(5):644-50) and Papaioannou I. et al (Expert Opinion on Biological Therapy, March 2012, Vol. 12, No. 3: 329-342) all of which are herein incorporated by reference.

Ribozymes can also function as inhibitors of Thrombin gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of thrombin mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNAs and ribozymes useful as inhibitors of Thrombin gene expression can be prepared by known methods. These include techniques for chemical synthesis such as, e.g., by solid phase phosphoramidite chemical synthesis. Alternatively, antisense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide, siRNA or ribozyme nucleic acid to the cells and preferably cells expressing thrombin. Preferably, the vector transports the nucleic acid within cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA or ribozyme nucleic acid sequences. Viral vectors are a preferred type of vectors and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in KRIEGLER (A Laboratory Manual,” W.H. Freeman C. O., New York, 1990) and in MURRY (“Methods in Molecular Biology,” vol. 7, Humana Press, Inc., Cliffton, N.J., 1991).

Preferred viruses for certain applications are the adenoviruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, nuclease (i.e. CrispR), siRNA, shRNA or ribozyme nucleic acid sequences are under the control of a heterologous regulatory region, e.g., a heterologous promoter. The promoter may be specific for the neural cells.

### Method of Preventing or Treating Pathological Conditions

The present invention further contemplates a method of preventing or treating inflammatory bowel disease (IBD) in a subject comprising administering locally in the Gut to the subject a therapeutically effective amount of a Direct Thrombin Inhibitor.

In a specific embodiment, the inflammatory bowel disease (IBD) is selected from the list consisting of Ulcerative colitis (UC) and Crohn's disease (CD).

Preferably, the inflammatory bowel disease (IBD) is Crohn's disease (CD)

In one aspect, the present invention provides a method of inhibiting inflammatory bowel disease (IBD) in a subject comprising administering a therapeutically effective amount of a Direct Thrombin Inhibitor.

By a “therapeutically effective amount” of a Direct Thrombin Inhibitor as described above is meant a sufficient amount of the antagonist to prevent or treat a inflammatory bowel disease (IBD). It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start with doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicine typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The invention also relates to a method for treating a inflammatory bowel disease (IBD) in a subject having a high level of thrombin in a biological sample (such as in blood, urinary or in feces sample) with a Direct Thrombin Inhibitor.

The invention also relates to Direct Thrombin Inhibitor for use in the treatment of a inflammatory bowel disease (IBD) in a subject having a high level of thrombin in a biological sample.

In a particular embodiment, the thrombin inhibitor is administered locally in the Gut.

The above method and use comprise the step of measuring the level of thrombin protein expression (protein or nucleic sequence (DNA or mRNA)) in a biological sample obtained from said subject wherein and compared to a reference control value.

A high level of thrombin is predictive of a high risk of having or developing a inflammatory bowel disease (IBD) (such as Crohn's disease (CD) and means that Direct Thrombin Inhibitor could be used.

Typically, a biological sample is obtained from the subject and the level of thrombin is measured in this tissue sample (ie gut). Indeed, decreasing thrombin levels would be particularly beneficial in those patients displaying high levels of thrombin.

### Pharmaceutical Compositions of the Invention:

The Direct Thrombin Inhibitor/inhibitor of thrombin gene expression as described above may be combined with pharmaceutically acceptable excipients, and optionally sustained-release matrices, such as biodegradable polymers, to form therapeutic compositions.

Accordingly, the present invention relates to a pharmaceutical composition comprising a Direct Thrombin Inhibitor according to the invention and a pharmaceutically acceptable carrier.

The present invention also relates to a pharmaceutical composition for use in the prevention or treatment of inflammatory bowel disease (IBD) (such as Crohn's disease (CD) comprising a Direct Thrombin Inhibitor according to the invention and a pharmaceutically acceptable carrier.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In therapeutic applications, compositions are administered to a patient already suffering from a disease, as described, in an amount sufficient to cure or at least partially stop the symptoms of the disease and its complications. An appropriate dosage of the pharmaceutical composition is readily determined according to any one of several well-established protocols. For example, animal studies (for example on mice or rats) are commonly used to determine the maximal tolerable dose of the bioactive agent per kilogram of weight. In general, at least one of the animal species tested is mammalian. The results from the animal studies can be extrapolated to determine doses for use in other species, such as humans for example. What constitutes an effective dose also depends on the nature and severity of the disease or condition, and on the general state of the patient's health.

In therapeutic treatments, the antagonist contained in the pharmaceutical composition can be administered in several dosages or as a single dose until a desired response has been achieved. The treatment is typically monitored and repeated dosages can be administered as necessary. Compounds of the invention may be administered according to dosage regimens established whenever inactivation of thrombin or PAR-1 is required.

The daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 10 mg/kg of body weight per day. It will be understood, however, that the specific dose level and frequency of dosage for any particular patient may be varied and will depend upon a variety of factors including the activity of the specific compound employed, the metabolic stability, and length of action of that compound, the age, the body weight, general health, sex, diet, mode and time of administration, rate of excretion, drug combination, the severity of the particular condition, and the host undergoing therapy.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The appropriate unit forms of administration include forms for oral administration, such as tablets, gelatine capsules, powders, granules and solutions or suspensions to be taken orally, forms for sublingual and buccal administration, aerosols, implants, forms for subcutaneous, intramuscular, intravenous, intranasal or intraocular administration and forms for rectal administration.

In the pharmaceutical compositions of the present invention, the active principle is generally formulated as dosage units containing from 0.5 to 1000 mg, preferably from 1 to 500 mg, more preferably from 2 to 200 mg of said active principle per dosage unit for daily administrations.

When preparing a solid composition in the form of tablets, a wetting agent such as sodium laurylsulfate can be added to the active principle optionally micronized, which is then mixed with a pharmaceutical vehicle such as silica, gelatine, starch, lactose, magnesium stearate, talc, gum arabic or the like. The tablets can be coated with sucrose, with various polymers or other appropriate substances or else they can be treated so as to have a prolonged or delayed activity and so as to release a predetermined amount of active principle continuously.

A preparation in the form of gelatin capsules is obtained by mixing the active principle with a diluent such as a glycol or a glycerol ester and pouring the mixture obtained into soft or hard gelatine capsules.

A preparation in the form of a syrup or elixir can contain the active principle together with a sweetener, which is preferably calorie-free, methyl-paraben and propylparaben as an antiseptic, a flavoring and an appropriate color.

The water-dispersible powders or granules can contain the active principle mixed with dispersants or wetting agents, or suspending agents such as polyvinyl-pyrrolidone, and also with sweeteners or taste correctors.

The active principle can also be formulated as microcapsules or microspheres, optionally with one or more carriers or additives.

Among the prolonged-release forms which are useful in the case of chronic treatments, implants can be used. These can be prepared in the form of an oily suspension or in the form of a suspension of microspheres in an isotonic medium.

The direct thrombin inhibitors according to the invention can be administered by any suitable route of administration. For example, direct thrombin inhibitor according to the invention can be administered by oral (including buccal and sublingual), rectal, nasal, topical (intracolic), pulmonary, vaginal, or parenteral (including intramuscular, intra-arterial, intrathecal, subcutaneous and intravenous) administration.

For the direct thrombin inhibitors according to the invention and in the preferred embodiment the DTI can be administered by oral (including buccal and sublingual), rectal or topical (intracolic) administration

The direct thrombin inhibitors of the present invention, may be formulated in a wide variety of oral administration dosage forms.

The term “preparation” is intended to include the formulation of the active compound with an encapsulating material as carrier, providing a capsule in which the active component, with or without carriers, is surrounded by a carrier, which is in association with it. Similarly, cachets and lozenges are included. Tablets, powders, capsules, pulls, cachets, and lozenges may be as solid forms suitable for oral administration. Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavours, stabilizers, and thickening agents. Aqueous suspensions can be prepared by dispersing the finely divided active component in water with viscous material, such as natural or synthetic gums, resins, methylcellulose, sodium carboxymethylcellulose, and other well-known suspending agents. Solid form preparations include solutions, suspensions, and emulsions, and may contain, in addition to the active component, colorants, flavours, stabilizers, buffers, artificial and natural sweeteners, dispersants, thickeners, solubilizing agents, and the like.

The direct thrombin inhibitors of the present invention of the present invention may be formulated for administration as suppositories. Typically, a low melting wax, such as a mixture of fatty-acid glycerides or cocoa butter is first melted and the active component is dispersed homogeneously.

### Use of a Food-Grade Bacterium

Use of a food-grade bacterium (or probiotic bacterium) to deliver direct thrombin inhibitors of the present invention especially polypeptide antagonist and more especially endogenous polypeptide allow to provides a safety and a good efficiency to deliver DTIs locally in the gut. Accordingly further object of the invention relates to a recombinant food-grade bacterium comprising a gene selected from a gene coding for a member of the serpin family proteins or an active fraction of a member of the serpin family proteins (AntiThrombin, Heparin Cofactor II, Protein C inhibitor and protease Nexin 1) for use in the treatment of a patient affected with an inflammatory bowel disease (IBD) said recombinant food-grade bacterium being administered locally in the gut of the patient to be treated.

Accordingly, in order to be administered directly to the intestine (gut), the recombinant food-grade bacterium according to the invention is administered to the IBD patient preferably orally (including buccal and sublingual administration), rectally or topically (intracolic administration),

Another aspect of the invention relates to a therapeutic composition comprising a recombinant food-grade bacterium as defined above.

Another aspect of the invention relates to a direct thrombin inhibitor (DTI) for use in the treatment of a patient affected with an inflammatory bowel disease (IBD) wherein a food-grade bacterium comprising a gene selected from a gene coding for a member of the serpin family proteins is used to deliver directly the serpins family polypeptide in the gut.

As used herein, the term “food-grade bacterium” denotes a bacterium that is widely used in fermented foods and possesses a perfect safety profile recognized by the GRAS (Generally Recognized As Safe) and QPS (Qualified Presumption of Safety) status in USA and European Community, respectively. Such bacterium can be safely in functional foods or food additives with allegations concerning maintain in good health and well-being or prevention of disease. As used herein, the term “probiotic bacterium” denotes a bacterium which ingested live in adequate quantities can exert beneficial effects on the human health. They are now widely used as a food additive for their health-promoting effects. Most of the probiotic bacteria are Lactic Acid Bacterium (LAB) and among them strains of the genera Lactobacillus spp. and Bifidobacterium spp. are the most widely used probiotic bacteria.

In a preferred embodiment, the gene according to the invention codes for the a member of the Serpin family such as AntiThrombin, Heparin Cofactor II, Protein C inhibitor and protease Nexin 1.

In a preferred embodiment, the food-grade bacterium strain according to the invention is a Lactococcus lactis strain or a Lactobacillus casei strain or a Lactococcus lactis htrA strain [Poquet et al., 2000] or a Lactobacillus plantarum strain or a Bifidobacterium longum strain. In a preferred embodiment, the food-grade bacterium strain according to the invention is a Lactobacillus casei strain.

### FIGURES

FIG. 1: Representative western-blot analysis (A) and relative abundance quantification (B) of thrombin protein expression in protein extracts from human colonic biopsies harvested from healthy control or Crohn's disease (CD) patients, and incubated for 1-h in PBS buffer. Bands with different molecular weights and corresponding to different forms of thrombin (Pro-thrombin, Meizothrombin, α-thrombin, β-thrombin, γ-thrombin) were detected (A), and quantified (B). Significant difference compared to controls were noted by ** for p<0.01, and *** for p<0.005, Student t test. (C) Immunohistochemistry for epithelial cell marker (EpCAM, epithelial cell adhesion molecule, left panels) and thrombin (middle panel) expression in human colonic biopsies harvested from healthy controls, Crohn's disease or Ulcerative Colitis patients. Scale bar is 50 μm.

FIG. 2: Colonic macroscopic damage score (A), colon thickness (B), aerobic (C) and anaerobic (D) bacteria translocated to mesenteric lymph nodes in mice that have received daily intracolonic administration of vehicle, thrombin (50 μl of 100 U/ml in saline), or boiled thrombin (same dose) for 10 days. Significant differences compared to thrombin-treated mice were noted by * for p<0.05, ** for p<0.01, and *** for p<0.001, ANOVA with Newman-Keuls post hoc test.

FIG. 3: Colon macroscopic damage score (A), wall thickness (B), bacterial translocation to mesenteric lymph nodes (C), Myeloperoxidase (MPO) activity (D), percent of apoptotic cells (E) and FITC-Dextran passage to blood (F) in PAR-1-deficient mice (PAR-1−/−), PAR4-deficient mice (PAR4−/−) and littermates (WT) 10 days after daily intracolonic administration of saline or thrombin (50 μl of 100 U/ml in saline). Significant differences compared to thrombin-treated mice were noted by ** for p<0.01, and *** for p<0.005, ANOVA with Newman-Keuls post hoc test.

FIG. 4: Effect of daily intracolonic administration of dabigatran (BIBR 953) on TNBS-induced colitis in rats (n=10), compared to uninflamed controls (CTR, that have received intracolonic PBS administration, n=6). A group of colitis rat (n=10) was treated daily with intracolonic administration of vehicle (saline). Disease activity index was recorded daily (A), and at sacrifice, colon thickness (B), macroscopic damage score (C), and myeloperoxidase (MPO) activity (D) were measured. Significant differences compared to CTR and vehicle-treated rats were noted * for p<0.05, ** for p<0.01 and *** for p<0.001. two-way ANOVA with Bonferroni post hoc test in (A) and ANOVA with Newman-Keuls post hoc test for (B-D).

FIG. 5: Effect of daily intracolonic administration of the thrombin inhibitor dabigatran (BIBR 953) on TNBS-induced colitis in rats (n=10) on fecal bleeding score. Colitis was induced by intracolonic administration of TNBS in 2 groups of rats, while one group of non-inflamed rats (Controls) received intracolonic administration of PBS. In the 2 colitis groups of rats, one received daily intracolonic administration of dabigatran, and the other received daily intracolonic administration of Saline (Vehicle). Fecal bleeding score was assessed by measuring the presence of blood in the feces (none: score 0; traces: score 1; evident blood: score 2). Significant differences between vehicle- and dabigatran-treated groups were noted * for p<0.05, using a t test.

FIG. 6: Effects of TNBS-induced colitis in wild-type (WT) and PAR4-deficient mice (PAR4−/−), compared to uninflamed controls (wild-type mice receiving intracolonic PBS instead of TNBS in 50% ethanol). Animal weight was recorded daily (A), and at sacrifice, colon thickness (B), macroscopic damage score (C) and myeloperoxidase (MPO) activity (D) were measured. Significant differences compared to vehicle-treated rats were noted by * for p<0.05, and by ** for p<0.01, two-way ANOVA with Bonferroni post hoc test in (A) and ANOVA with Newman-Keuls post hoc test for (B-D)

FIG. 7: (A) Exposure to High thrombin causes increased adhesive properties of human mucosal biofilms to human intestinal epithelial cells (B) Exposure to High thrombin causes increased adhesive properties of adherent invasive Escherichia coli (LF82) biofilms to human intestinal epithelial cells (C) Exposure to High thrombin of adherent invasive Escherichia coli (LF82) biofilms causes increased invasion of Caco-2 cell line.

### EXAMPLE 1

Material & Methods

Tissue Collection and Patients—

Human colonic tissue collection received ethical approval from the French Ethic Committee (Identifier: NCT01990716). Patients received written and verbal information about the study and signed informed consent before the enrollment in the study. Colonic tissue samples were obtained from diagnosed CD and UC patients undergoing colonoscopy at the Toulouse Hospital, Gastroenterology Pole. Healthy control samples were from individuals undergoing colon cancer screening who were negative for cancer diagnosis and otherwise healthy. Upon collection, fresh colonic biopsies were either immediately embedded in optimal cutting temperature (OCT), snap-frozen in dry ice and stored at −80° C. for further in situ zymography or immunohistochemistry analysis, or stored at −80° C. in 400 μL of RP1 buffer (Macherey-Nagel). Tissue protein extraction from colonic biopsies was performed by using the RNA/Protein Nucleospin Kit, according to manufacturer's instructions (Macherey-Nagel). Upon protein quantification by using the Pierce Protein BCA Assay Kit (according to/adapted to manufacturer's instructions (Thermo Scientific/Macherey-Nagel), protein content was adjusted to 5 μg/μL in protein solving buffer (PSB) supplemented with the reducing agent tris(2-carboxyethyl)phosphine (PSB-TCEP; Macherey-Nagel). Samples were heated at 95° C. for 5 min, before being used for further western-blot analysis. Measurements of thrombin activity—Serine-protease activity was quantified in colonic washes from rat with BOC-Val-Pro-Arg-amino-4-methylcoumarin hydro-chloride (200 μM, Sigma-Aldrich B9395) as substrate in 50 mM Tris, 10 mM CaCl2, 150 mM NaCl, pH=8.3. Initial velocity was calculated by the change in fluorescence (excitation: 355 nm, emission: 460 nm), measured over 15 min at 37° C. using a Varioskan Flash microplate reader (Thermo Fisher Scientific). Thrombin specific activity was detected by pre-incubating samples with Dabigatran (1 μM, selleckchem S2196) for 10 min at 37° C. prior starting enzymatic reaction. Thrombin activity was detected in tissues by in situ zymography. Cryo-sections of colonic human tissue were rinsed with 2% Tween in PBS, and 50 μl per slice of 400 μM BOC-Val-Pro-Arg-amino-4-methylcoumarin hydro-chloride were applied for 4-hours at 37° C. To confirm specific thrombin activity on tissues, frozen sections were pre-incubated with 10 μM dabigatran for 30 min and incubated with substrate buffer plus 10 μM dabigatran. Nuclei were counterstained using 4′,6-diamidino-2-phenylindole. Sections were visualized with LSM 710 confocal microscope. Representative images were obtained from random acquisition of minimal 4 different fields. Imaging analysis was performed with Zen 2009 software (Carl Zeiss). Western-blot—Fifty μg (10 μL) of protein per sample were loaded per lane, run into 4-20% Mini-Protean TGX precast gels (BioRad, 456-1085) and transferred onto Nitrocellulose membrane by using the Trans-Blot Turbo System (BioRad). The membrane was blocked for 1 h in PBS supplemented with 1% bovine serum albumin and 0.05% Tween-20, and then incubated overnight in the same buffer complemented with the goat IgG anti-thrombin (1:200; K-20; sc-16972, Santa Cruz Biotech). Membrane was then washed and incubated for 1 h with secondary antibody donkey anti-goat IgG conjugated to HRP (1:50,000; Jackson Immunoresearch). Immunoblots were resolved by using the kit ECL Prime, according to instructions (GE Healthcare). Membranes were visualized by using a Chemidoc XRS (Bio-Rad). Hence, membranes were striped and subjected to the same steps to immunodetect β-actin (1:10,000, REF), followed by incubation with anti-mouse IgG conjugated to HRP (V805A, Promega). The molecular weight of bands of interest and the relative abundance to p-actin were estimated with the software ImageLab (version 5.0, build 18; Bio-Rad). Data are expressed as fold change relative to the control group.

RT-PCR—

Transcription levels of mucosal thrombin, encoded by F2 gene, have been assessed by scrapping the mucosa of distal colons of mice. Total RNA was extracted using the Qiagen RNeasy kit according to the manufacturer's instructions (Qiagen). Three μg of mRNA was reversely transcribed into cDNA (iScript cDNA synthesis kit, Biorad), and the qPCR reaction was performed on the LightCycler 480 (Roche). The expression levels of rat F2 (Forward: AGGCCTGACATCAACTCCAC (SEQ ID N^{o} 1); Reverse: TTGGAACCTCTTGAGCGAGG (SEQ ID N^{o} 2) were normalized to mRNA expression of housekeeper gene Glyceraldehyde 3-phosphate dehydrogenase (GAPDH, Forward: CAAGGTCATCCATGACAACTTTG; (SEQ ID N^{o} 3) Reverse: GGGCCATCCACAGTCTTCTG (SEQ ID N^{o} 4)), and fold change were calculated using the 2-ΔΔCt method.

Immunohistochemistry—

As described above, human colonic biopsies were cryopreserved in optimal cutting temperature (OCT) compound and sectioned at 6 μm thickness. Slides were thawed at room temperature for 20 min and incubated with blocking solution (1% BSA, 0.3% Triton X-100, PBS 1×) for 1 h. They were then incubated in the same solution complemented with the anti-thrombin described for Western-blot analysis (1/250 dilution) overnight. The secondary antibody used for detection was an Anti-Goat Alexa-555 from Life Technologies (A211432) used at 1/1000 dilution as previously described⁹. Counterstaining of nuclei was performed using 4′,6-diamidino-2-phenylindole from Invitrogen France. EpCAM (epithelial cell adhesion molecule) staining was used as an epithelial marker (anti-EpCAM, ab71916, Abcam). Representative images were obtained from random acquisition of 4 different fields, and images were acquired using a Leica LSM710 confocal microscope. FIJI freeware was used for final image mounting (v 1.51).

Animals—

Wistar rats (150-200 g) and C57Bl6 mice (8-weeks old) were purchased from Janvier laboratories, Saint-Quentin Fallavier (France). PAR-1-deficient mice (PAR-1^{-/-}) and littermates (C57BL/6 background) were originally provided by Johnson & Johnson Pharmaceutical Research and Development. PAR4-deficient mice (PAR4^{-/-}) and littermates (C57BL/6 background) were originally provided by Shaun Coughlin, University of San Francisco, California. PAR-1^{-/-}, PAR4^{-/-} and littermates were all bred at the University of Calgary, and were housed in a temperature-controlled room and had free access to food and water. The Animal Care Committee of the University of Calgary approved all experimental protocols for experiments using PAR-deficient mice. For all the other animal experimentation, they were performed in Toulouse, under the approval of the Animal Care and Ethics Committee of US006/CREFRE (CEEA-122, APAFIS #7762-20161125092278235).

Intracolonic Administration of Thrombin—

Thrombin (specific activity 2000 NIH units/mg protein, T6884, Sigma) was administered daily for 10-days intracolonically daily (5 U/mouse in 50 μl in saline, 0.9% NaCl) under light anaesthesia (3% isoflurane) in PAR-1^{-/-}, PAR4^{-/-}, littermates and in mice purchased from Janvier laboratories. Controls received heat-inactivated thrombin (same dose, 10 minutes at 100° C.) or saline alone. After intracolonic instillation, mice were kept upside-down for 2-min to allow colonic diffusion of the administered solution. After 10-days, mice were sacrificed by cervical dislocation and inflammation parameters were measured.

Colitis Models and Inflammation Parameters—

TNBS colitis was induced in fasted rats as previously described^{20,21}. Animals were housed in ventilated cages, acclimatized for one week before experiment started and beddings were mixed twice. Briefly, TNBS was prepared at a final concentration of 120 mg/ml in 50% ethanol in saline and was kept in the dark before use. A catheter was inserted at 8 cm from the anus and 30 mg of the TNBS solution was instilled through the catheter into the lumen in a final volume of 250 μl/rat. For TNBS colitis induction in mice, fasted animals were lightly anesthetized with halothane. A polyethylene catheter was inserted intracolonically 4 cm from the anus and TNBS (2 mg/mouse in 100 μl) dissolved in a solution of saline plus 40% ethanol was pushed into the catheter. At time of the sacrifice, colonic tissues were harvested. Before opening the colon, luminal washes with a PBS solution were performed in order to measure thrombin activity in those washes. Colons were then cut opened. Macroscopic damage scores were evaluated as previously described⁹, by a skilled observer unaware of the treatments. When observed, the following parameters were given score of 1: hemorrhage, edema, stricture, ulceration, fecal blood, mucus, and diarrhea. Erythema was scored a maximum of 2 depending on the length of the area being affected (0, absent; 1, less than 1 cm; and 2, more than 1 cm). Adhesion was scored based on its severity (0, absent; 1, moderate; and 2, severe). Colon thickness was measured using an electronic caliper (Mitutoyo, Mississauga, Canada, resolution 0.01 mm). MPO activity was measured as an index of granulocyte infiltration as previously described. Tissue samples were homogenized in a solution of 0.5% hexadecyltrimethylammonium bromide dissolved in phosphate buffer solution (pH 6.0) for 1 min. Homogenized tissues were centrifuged at 13 000 g for 2 min. Supernatants were added to a buffer supplemented with 1% hydrogen peroxide, and O-dianisidine dihydrochloride solution. Optical density readings were taken for 1 min at 30 s intervals at 450 nm²². Results were normalized to total protein content and expressed as OD_{450 nm}/min/μg/ml. For bacterial translocation assays, mesenteric lymph nodes were collected aseptically, weighed and homogenized before being plated at 37° C. in Columbia blood agar (BD Biosciences) for 24 h for aerobes, and for 48 h for anaerobes left in anaerobic jars⁹. For permeability assays, mice received orally 4-kDa FITC Dextran (Sigma Aldrich) at the dose of 600 mg/kg body weight in PBS, and blood was collected 1-h after. Fluorescence for FITC-Dextran presence in serum was measured by spectrophotometry (excitation 485 nm, emission 528 nm)²³. Apoptosis was measured by TUNEL (terminal deoxynucleotidyl transferase dUTP nick end labeling) staining in mouse colonic tissues as previously described^{24,25}. Five random fields were captured per tissue section and percent of apoptotic cells was determined by an observer unaware of the treatments.

Thrombin and PAR-1 Inhibition During Experimental Colitis in Rats—.

Rats were randomly divided into control group without colitis (intracolonic administration TNBS vehicle PBS, 100 μl, n=6) and two TNBS-induced colitis groups (intracolonic administration of TNBS 120 mg/ml in 50% ethanol), in which one was administered intracolonically dabigatran (100 μl, 5 μM dabigatran BIBR953, n=10), and the other one was administered drug vehicle (100 μl, 0.1% DMSO, n=6). Intracolonic administrations of dabigatran or vehicle were performed during 4 days. Animals were euthanized by lethal dose of pentobarbital. Disease activity index was evaluated daily throughout the experimentation based on i) fecal consistency (0 for normal feces, 1 for soft feces, 2 for diarrhea), ii) rectal bleeding (0 for negative, 1 for positive, 2 for gross bleeding), iii) prolapses (0 for negative, 1 for positive, 2 for gross prolapses), and iiii) the presence of abdominal mass (0 for negative, 1 soft abdominal mass, 2 hard abdominal mass). In order to test the effects of the PAR-1 antagonist Vorapaxar on TNBS-induced colitis, TNBS colitis rats received daily oral treatments with Vorapaxar (2.5 mg/kg) or vehicle (PBS), starting 1 h before the TNBS intracolonic administration.

Statistics—

Statistical comparisons among groups were performed according to dataset structure using either unpaired Student or Mann-Whitney test, one-way analysis of variance (ANOVA) followed by the Student-Newman-Keuls multiple comparison test, and two-way ANOVA followed by Bonferroni multiple comparison test. Outliers were identified using ROUT test set at Q=1%. Graphic representation and statistical analyses were performed using GraphPad Prism (v6, La Jolla, USA). Data are expressed as mean±standard error means, and n of groups appear in figures. Probability (p value) was considered statistically significant for p<0.05.

Results

—Mucosal Thrombin is Up-Regulated Upon Intestinal Inflammation—

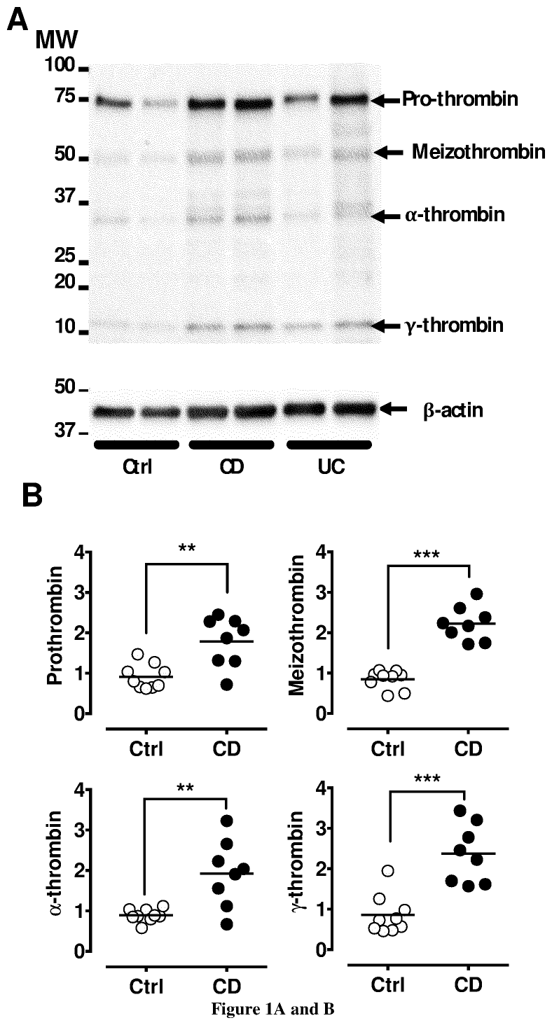

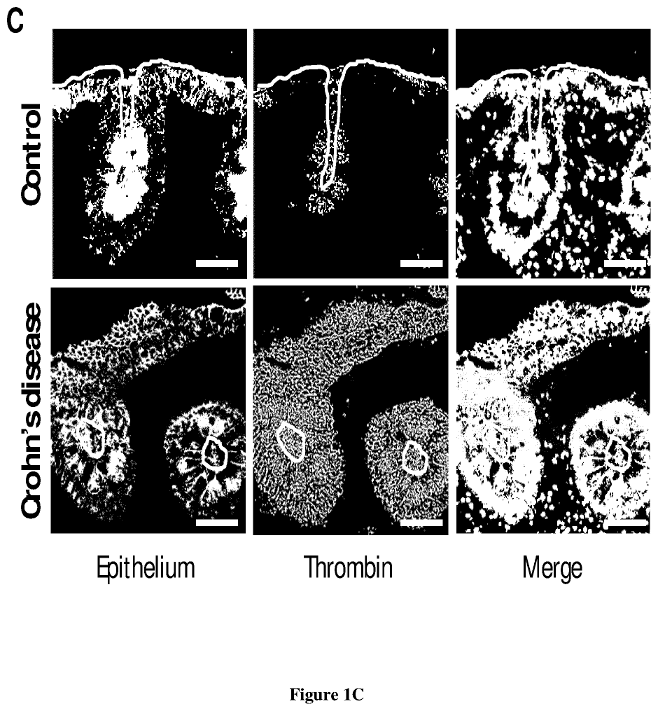

We and others have previously reported that thrombin activity released by tissues from IBD patients was significantly increased compared to tissues from healthy controls^{5,10,11}. Here, we wanted to confirm that human intestinal tissues could indeed produce the thrombin protein, further identifying the presence of the different active and inactive forms of thrombin. We performed Western-Blot analysis on tissue extracts of human biopsies (FIG. 1A). Under reducing conditions, thrombin bands²⁶ corresponding to the pro-thrombin form (˜70 kDa), along with bands corresponding to active forms of thrombin: meizothrombin (˜53 kDa), alpha-thrombin (˜32 kDa), gamma-thrombin (˜15 kDa) were detected in biopsy protein extracts from healthy controls and CD (FIG. 1A). Quantification of the detected bands revealed that pro-thrombin, alpha-thrombin, meizothrombin and gamma-thrombin forms were all significantly up-regulated in biopsies from CD patients, compared to healthy controls (FIG. 1B). Although the different forms of thrombin protein were upregulated as well in UC, it was less marked compared to CD (data not shown). Immunohistochemistry showed that thrombin expression was associated with intestinal epithelial cells and was present at the mucosal surface (in the lumen) of both healthy controls and CD patient tissues (FIG. 1C). Staining for thrombin protein expression was stronger in tissues from CD patients, compared to healthy controls (FIG. 1C). We then used in situ zymography to get more insights on the localization of thrombin's activity in human colonic tissues from IBD patients (2 examples of CD patient tissues, Supplementary FIG. 1), using the fluorogenic thrombin substrate VPR-AMC. We observed that the thrombin activity detected in tissues from CD patients was elevated in the mucosa and the submucosa, showing a strong association with the epithelium (data not shown). The detected tissue activity was inhibited by incubation with Dabigatran, a thrombin inhibitor classically used therapeutically (BIBR 953), (data not shown). Similar response was obtained using another thrombin inhibitor (lepirudin, not shown), hence confirming the specificity of thrombin activity detected in situ on frozen colonic tissues.

In a rat model of CD, where colitis was induced by the intra-colonic administration of TNBS in 50% ethanol, we measured thrombin activity in colonic washes, 7-days after the induction of colitis. This activity was compared to the activity detected in colonic washes of rats that have received saline (0.9% NaCl) intracolonic administration. Thrombin specific activity was confirmed using the direct thrombin inhibitor Dabigatran (not shown). Like in CD patients, we observed that inflamed rat colons released significantly higher thrombin activity compared to non-inflamed rat colons (9.1-times increase in TNBS colitis versus healthy controls, data not shown). Further, we observed that thrombin mRNA expression was significantly up-regulated by 3-times in TNBS-treated rat colons, compared to saline-treated rat colons (data not shown).

Taken together, these results showed that thrombin is present both as a pro- and an active form in mucosal tissues, with a prominent expression and activity associated with the mucosa. Mucosal thrombin activity is up-regulated in IBD. The TNBS rat model of IBD reproduces this increased thrombin expression and activity.

—Luminal Thrombin Up-Regulation Recapitulates Mucosal Inflammation—

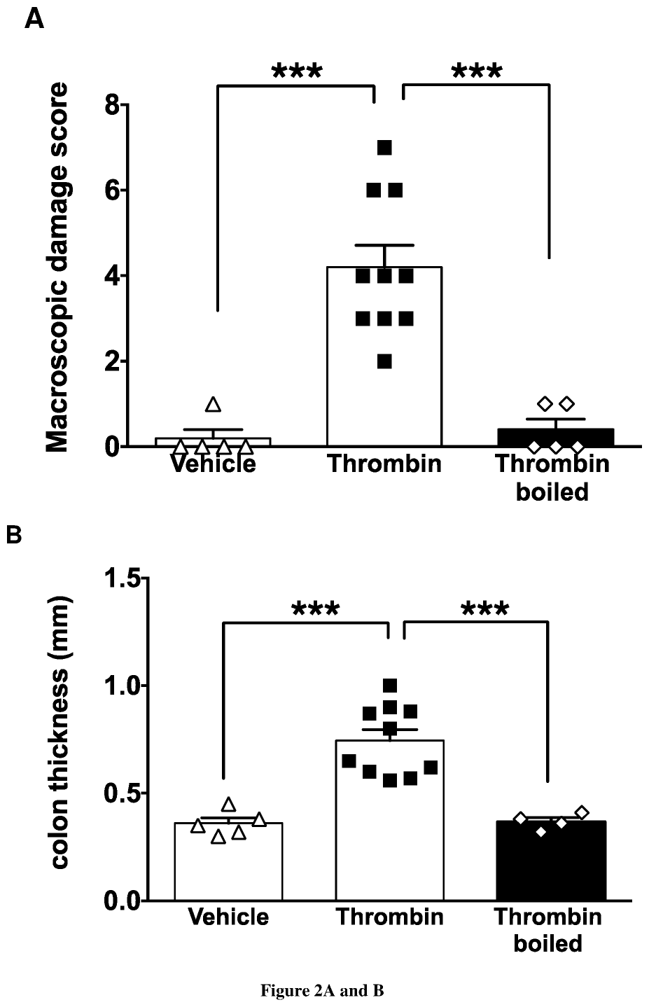

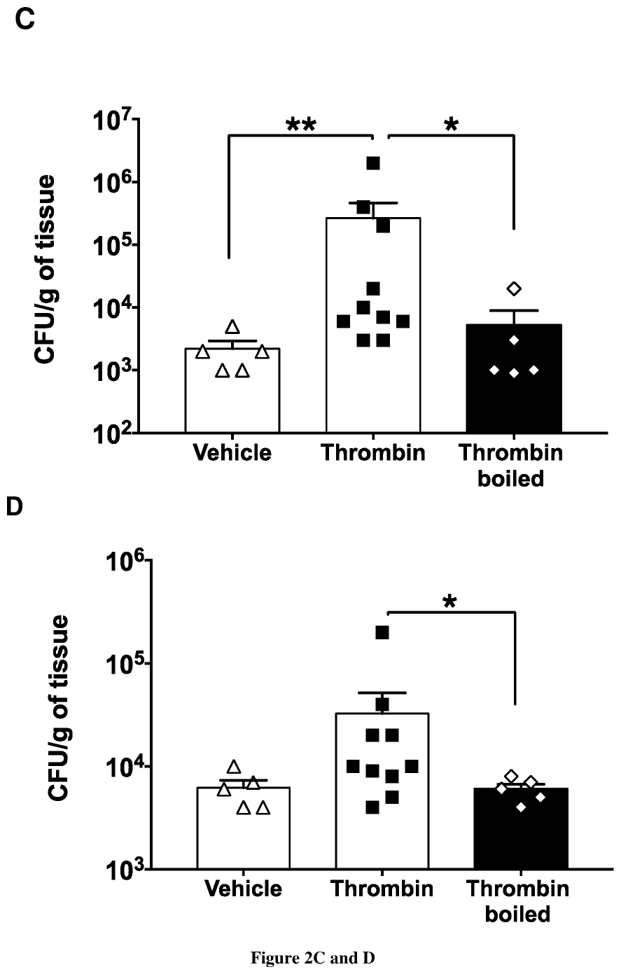

Since thrombin activity and expression were up-regulated in the lumen of inflamed colon (human and rats), we hypothesised that thrombin itself might lead to an inflammatory response in colon tissue. We have previously determined that the physiological range of thrombin activity in healthy colon lumenal samples is in the range of 20 U/ml per day in humans and 1.5 U/ml per day in mice⁹. Thrombin activity was 5 times and 98 times increased in tissues from UC and CD patients respectively⁵. Therefore, to evaluate the impact of elevated luminal thrombin levels on the mouse colon in vivo, we chose a concentration equivalent to that observed in CD patients (100 U/ml). We observed that daily intracolonic administration of thrombin (50 μl of 100 U/ml per mouse), but not boiled thrombin (same dose) caused macroscopic damage (FIG. 2A), and a significant increase of colon thickness (FIG. 2B), compared to vehicle (saline)-administered mice. When administered incracolonically, active thrombin also caused the translocation of aerobic bacteria into mesenteric lymph nodes, suggesting a disrupted intestinal barrier function (FIG. 2C). Translocation of anaerobic bacteria was not significantly different in thrombin-administered mice, compared to vehicle or boiled thrombin intracolonic administration (FIG. 2D).

—PAR Activation is Involved in Thrombin-Induced Intestinal Inflammation—

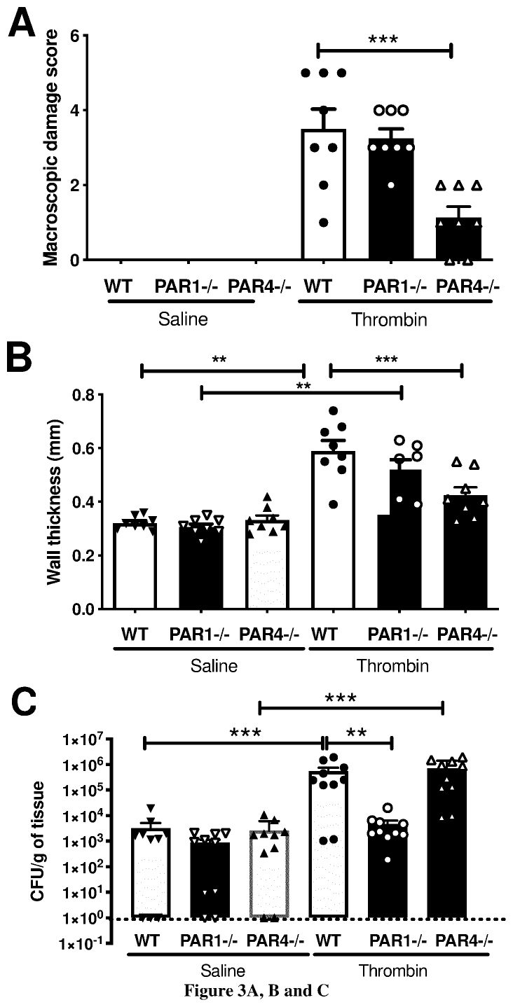

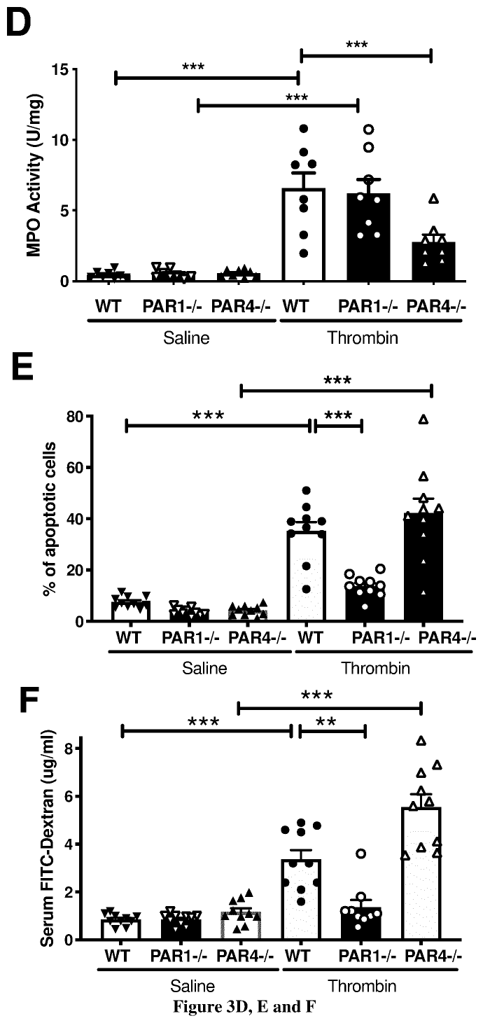

We then investigated whether the pro-inflammatory effects of thrombin administered into the colon of mice were dependent on the two signaling thrombin receptors: PAR-1 and PAR4. We administered thrombin intracolonically as described above, in wild-type as well as in PAR-1- or PAR4-deficient mice. In accordance with the data described above, we observed that in wild-type mice that thrombin caused signs of inflammation characterized by an increased macroscopic damage score, increased colonic wall thickness and increased bacterial translocation (FIG. 3A, B, C). In addition, we followed MPO activity, apoptosis and permeability (as measured by the passage of FITC dextran) (FIG. 3D, E, F). All of these inflammatory parameters were significantly increased in wild-type mice that received an intracolonic administration of thrombin (100 U/ml per day per mouse) compared to mice that received intracolonic saline alone (FIG. 3). PAR-1-deficient mice did not show increased bacterial translocation, apoptosis or permeability in response to intracolonic thrombin (FIG. 3C, E, F), but still exhibited significant macroscopic damage, increased colonic wall thickness and elevated MPO activity (FIG. 3A, B, D). In contrast, thrombin-induced damage score, increased wall thickness and elevated MPO activity were significantly reduced in PAR4-deficient mice (FIG. 3A, B, D), whereas the thrombin-induced increase in bacterial translocation, apoptosis and increased permeability were unchanged in the PAR4-null mice (FIG. 3C, E, F). Taken together, these data showed that both PAR-1 and PAR4 activation are involved in the inflammatory response to intracolonically administered thrombin, these two PARs appear to regulate different responses triggered by thrombin.

—Thrombin-, PAR-1—but not PAR4-Inhibition Protected Against TNBS-Induced Colitis—

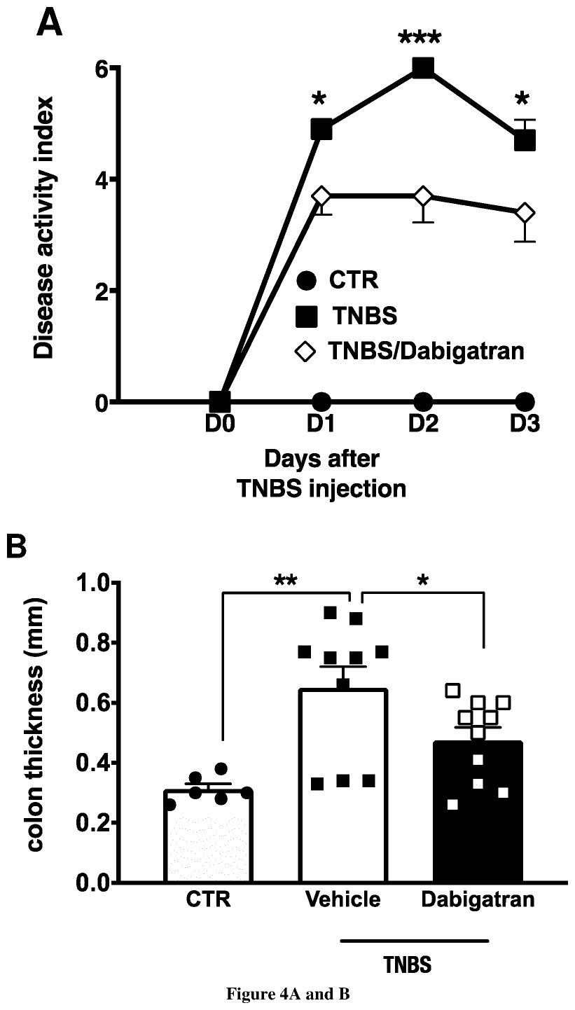

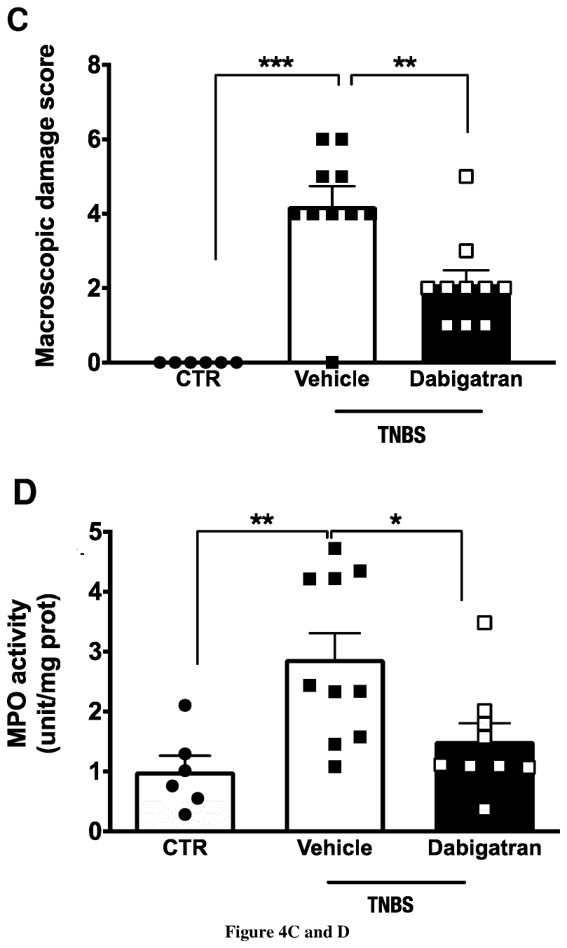

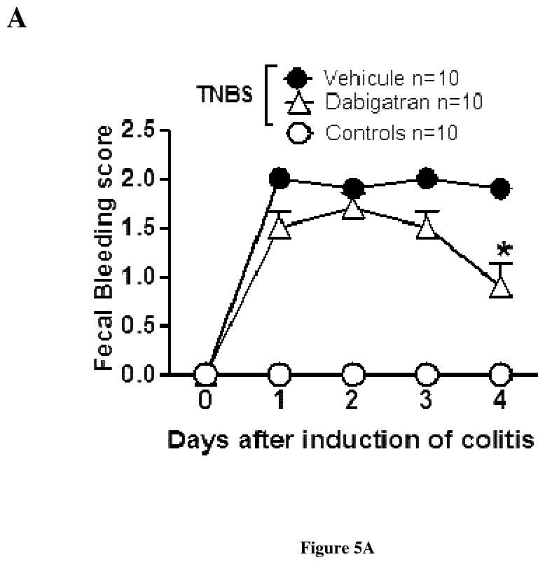

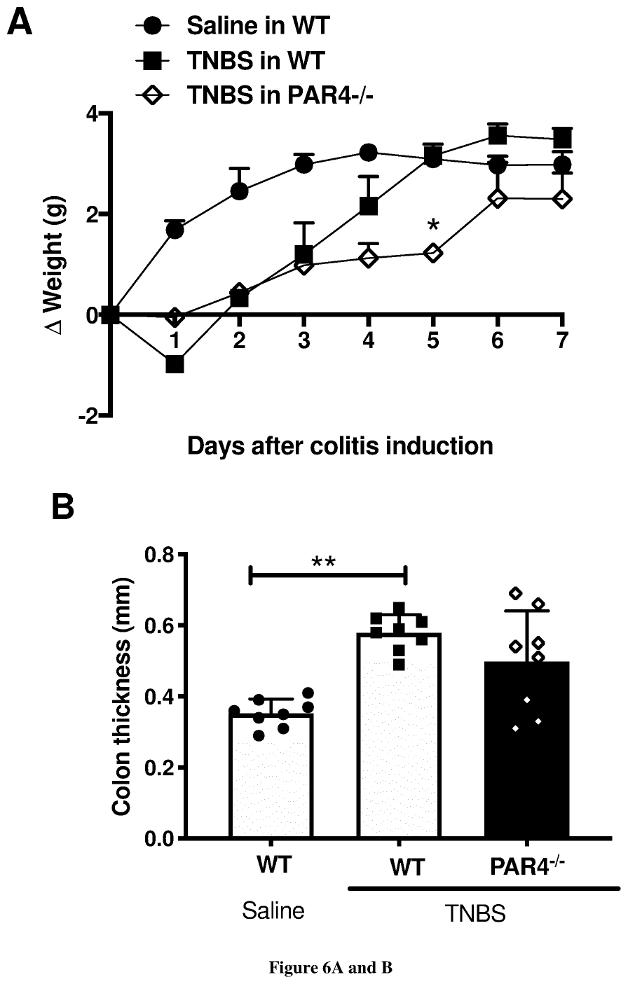

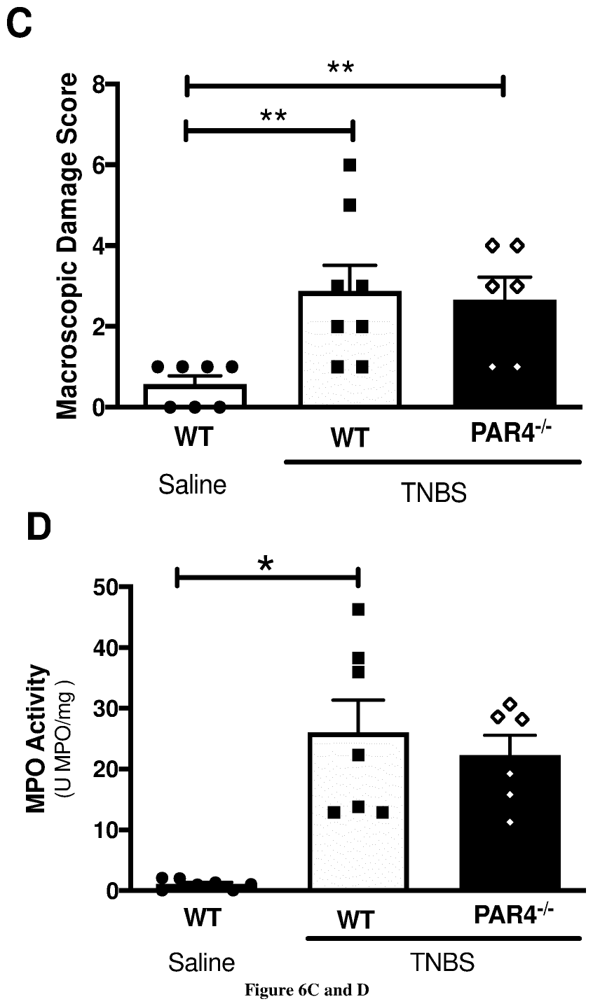

Having observed that the presence and activity of thrombin are drastically increased in colons of IBD patients and in IBD models, and that intracolonic thrombin itself can induce signs of inflammation, we next investigated the net contribution of thrombin to disease in a rat model of colitis induced by the intracolonic administration of TNBS. Since we wanted to focus on the contribution of mucosal thrombin to colitis, it was important to inhibit thrombin activity locally, in colonic tissues rather than inhibiting systemic thrombin activity. Therefore, after the induction of colitis in rats by the intracolonic administration of TNBS, rats were treated daily by an intracolonic administration of the direct thrombin inhibitor Dabigatran (BIBR 953). TNBS-induced colitis in rats caused significant weight loss, increased colon thickness, macroscopic damage score and increased MPO activity (FIG. 4). Daily intracolonic administration of the direct thrombin inhibitor Dabigatran protected rats from TNBS colitis, reducing significantly all these inflammation parameters (FIG. 4). Interestingly anti-thrombin treatment with Dabigatran reduced significantly blood in the feces in the TNBS induce colitis (FIG. 5). This result is counterintuitive this one could have expected the opposite: an increase of bleeding under anti-thrombin treatment. This result suggests that increased thrombin activity in the colon of TNBS-treated rats participates to intestinal inflammation. Because there are no selective and easily bioavailable PAR4 antagonists for in vivo use, in order to investigate the contribution of PAR4 in causing colitis, we used PAR4-deficient mice in which we induced colitis with TNBS. Seven days after receiving an intracolonic administration of TNBS, wild-type mice showed significant damage, increased colon thickness and MPO activity (FIG. 6). Weight loss associated with colitis in wild-type mice was significant at day 5 after the induction of colitis (FIG. 6). None of these inflammation-related parameters were different in PAR4-deficient mice compared to wild type mice after the induction of TNBS colitis, suggesting that PAR4 contributes only marginally in generating inflammation in that model (FIG. 6).

Discussion

While the implication of the hemostatic system in IBD has been investigated through the presence of hemostatic factors^{13,15,16}, or the effects of indirect thrombin inhibitors such as Heparin on disease parameters¹⁷⁻¹⁹, no study has clearly defined the role of local thrombin or other thrombosis factors in intestinal tissues. Is thrombin pro-inflammatory at the doses that are found in IBD patient tissues? What thrombin-induced signaling pathways would be involved in intestinal inflammation? Is thrombin a target for IBD treatment? Through the present invention, we aimed at providing some answers to such questions.

In a previous study, we have reported that thrombin activity was 100 times increased in tissues from Crohn's disease patients, compared to tissues from healthy controls⁵. What are the consequences for tissue homeostasis of such amounts of active thrombin in the colon? We demonstrated here in mice that exposing colon mucosa to equivalent concentration of thrombin activity that is found in tissues of Crohn's disease patients, caused signs of inflammation characterized by mucosal damage and increased permeability as observed by bacterial translocation. Further, we demonstrated in a rat model of colitis that thrombin activity and expression were increased in the same proportions as in Crohn's disease patient tissues (data not shown). In that particular model, we demonstrated that local inhibition of thrombin activity achieved by the intralumenal administration of the direct thrombin inhibitor Dabigatran was protective, inhibiting both mucosal damage, granulocyte infiltration and favoring weight gain (FIG. 4). Taken together, these results clearly demonstrated the deleterious effects of high doses of thrombin and reported the protection achieved by local thrombin inhibition in a model of IBD. Therefore, these results clearly point to mucosal thrombin inhibition as a possible target to treat intestinal inflammation and to reduce some inflammatory signs.

Thrombin exists under different forms. The inactive prothrombin form of ˜70-kDa is proteolytically converted to active α-thrombin (˜32-kDa), which may be further hydrolyzed to β- (˜28-kDa) and γ-thrombin (˜15-kDa). In addition, a transient form of thrombin (meizothrombin, ˜53-kDa) is an intermediate form of prothrombinase catalyzation²⁷. All the cleaved forms of prothrombin retain catalytic activities, and have been demonstrated to play important physiological roles, including the activation of PARs²⁷⁻²⁹. We detected in protein extracts of human colonic biopsies prothrombin, meizothrombin, α-thrombin and γ-thrombin. All of them, including the inactive prothrombin form were upregulated in Crohn's disease patients, while an upregulation of pro-thrombin, meizothrombin and γ-thrombin was observed in UC patient biopsies. At this stage, it is impossible to know which form of active thrombin might play a prominent role in inflammatory processes, and particularly in IBD. In experiments where thrombin was administered intracolonically and induced signs of inflammation, human α-thrombin was used. However, we cannot conclude that all the inflammatory signs observed are exclusively due to α-thrombin, because upon its administration into the colon of mice, this form of thrombin might well undergo autolytic degradation into β- or γ-thrombin. Our results though, demonstrated that catalytically active thrombin was involved in the generation of inflammation parameters. Indeed, inhibition of thrombin activity by local delivery of Dabigatran protected from TNBS-induced colitis. The increased presence of all active forms of thrombin associated with IBD is also in favor of a role for proteolytically active thrombin in the colon of IBD patients.

Here, we also demonstrated that the pro-inflammatory effects of thrombin were mediated through the activation of both PAR-1 and PAR4, acting on different parameters of inflammation. Intracolonic administration of active α-thrombin provoked several inflammatory signs such as macroscopic damage, granulocyte infiltration, and increased wall thickness, all of which were significantly reduced in PAR4⁻ but not in PAR-1⁻ deficient mice. Indeed, previous studies have reported that PAR4 activation, but not PAR-1 activation was responsible for thrombin-induced leukocyte rolling and adhesion in rat mesenteric venules³⁰. Another report demonstrated in a model of paw inflammation, that edema was formed as a consequence of PAR4-induced neutrophil recruitment³¹, thereby linking these two inflammatory parameters: edema and leukocyte recruitment, to PAR4 activation. Our present observations are in complete agreement with these studies, as they are pointing to PAR4-dependent mechanism of action responsible for thrombin-induced granulocyte recruitment and increased wall thickness (most likely due to edema). Other thrombin-induced inflammatory signs, included increased permeability, bacterial translocation and increased apoptosis, which were all dependent on the activation of PAR-1, but not PAR4. Previous reports have indeed identified that PAR-1 activation on intestinal epithelial cells induced apoptosis leading to increased intestinal permeability^{24,25,32}. Therefore, in our study it is tempting to consider that the thrombin-induced increased intestinal permeability and bacterial translocation were due to PAR-1 activation on intestinal epithelial cells, and potentially to an overactivation of apoptotic pathways.

Using animal models of IBD, we observed that thrombin was overexpressed and overactivated, and PAR-1 inhibition appeared as a better target than PAR4 to reduce colitis in this TNBS model. Several possible explanations could be proposed. In the TNBS model, other proteases such as elastase are also hyperactive^{7,33} which could disarm PAR4, by cleaving its N-terminal domain up-stream from the canonical thrombin activation site, thereby becoming a non-signalling receptor³⁴⁻³⁷. The forms of thrombin that are released in the TNBS colitis model might have a preferred PAR-1 substrate, compared to PAR4. It is known that α-thrombin has a better affinity for PAR-1 cleavage, while β- and γ-thrombin have a good affinity for PAR4 but not for PAR-1 cleavage²⁸. Finally, active thrombin present in colonic tissues upon TNBS colitis might not be in the vicinity of PAR4-expressing cells, but rather close to PAR-1-expressing cells.

The source of thrombin at mucosal surfaces is an intriguing question in the context of IBD. Tissue damage and ulcers are common features of IBD. Concomitant damage to blood vessels induce the activation of the clotting system and the associated on-site activation of circulating prothrombin. Part of the increased thrombin activity detected in IBD patient tissues might therefore originate from prothrombin recruitment and activation at injured sites. However, a recent study reports that intestinal epithelium is able to constitutively produce active thrombin⁹. Thrombin epithelial expression was shown to be under the control of microbial presence in the intestine. A dysregulated epithelial production of active thrombin due to abnormalities in the mucosal gut biofilm could well be associated with IBD. In the present study, in situ zymography (Supplementary FIG. 1) and immunostaining (FIG. 1C) determined that a large amount of thrombin activity detected in tissues from Crohn's disease patients, was associated with the epithelium, giving credit to this hypothetical source of active thrombin in the context of IBD.

A major finding of this study is that too much mucosal thrombin is detrimental in IBD, and can participate to inflammatory signs, through the activation of PARs. Inhibition of thrombin activity could thus be considered for the treatment of IBD¹⁷⁻¹⁹. In agreement with our findings, previous studies have reported in tissues from IBD patients the formation of microthrombi in bowel capillaries, suggesting that prothrombotic status could be a determinant factor in IBD pathogenesis¹⁵. Increased morbidity and mortality associated with hyperthrombosis in IBD can be manageable by available drugs³⁸. However the bleeding associated with IBD renders the guidance for anticoagulant thromboprophylaxis complicated in clinical practice³⁸. In addition, it was recently demonstrated that epithelial thrombin was necessary to preserve mucosal homeostasis, in part by maintaining spatial segregation between mucosal biofilms and host epithelium⁹. Although drugs are available in human for thrombin inhibition, in the context of IBD the side effects of such approach on bleeding and on biofilm containment might be difficult to manage. Targeting downstream events from high thrombin activity might be a better option for IBD. In the present study, we showed that PAR-1 activation plays a major role in the pro-inflammatory effects of thrombin, and could therefore constitute a more appropriate target for IBD treatment. The compound we tested here (Vorapaxar) is a PAR-1 antagonist that has been approved for human's use for cardiovascular indications³⁹. Considering our preclinical results with this compound, phase-II clinical studies in IBD patients could therefore be rapidly envisioned, particularly in Crohn's disease patients who seemed to present the strongest thrombin activity.

### EXAMPLE 2 THROMBIN EFFECT ON MUCOSA-ASSOCIATED MICROBIOTA

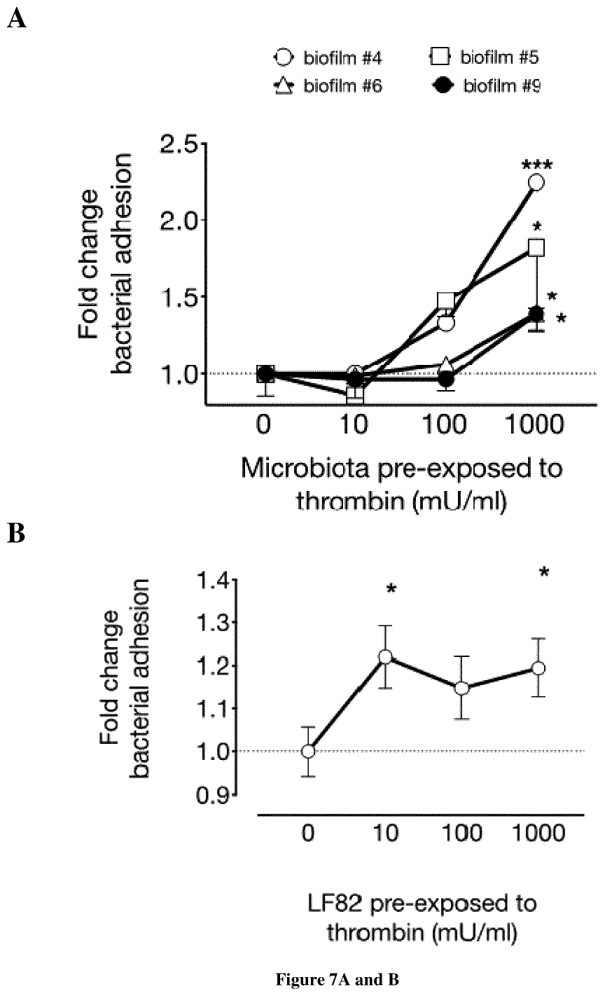

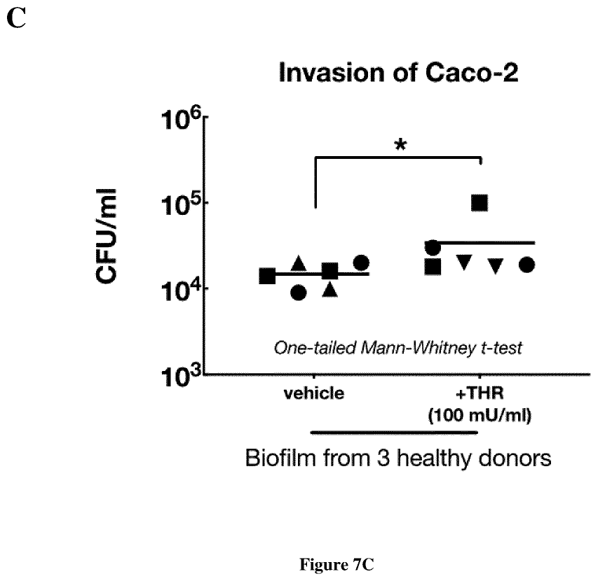

Methods for FIG. 7. Panel A: Mucosa-associated microbiota was extracted from 4 healthy human colon biopsies (collected in Toulouse Hospital under ethic approval CODECOH Colic-DC2015-2443), under anaerobic condition, and were seeded into the Calgary Biofilm Device (Innovotech, Canada) to develop a multispecies biofilm. After 48 hours of incubation (37° C., anaerobic environment), biofilms were exposed to various concentrations of human thrombin (0, 10, 100 and 1000 milliunit/ml). After 24 hours, dispersed bacteria from thrombin-treated biofilms were collected and apically exposed to human epithelial monolayers on 12-wells plates (Caco2, ATCC HB37). Bacterial adhesion to epithelial surface was assessed. Briefly, after 90 minutes of co-culture, cells were vigorously washed in PBS, and lyzed in Triton X100 0.1% for 10 minutes, after which live cell bacteria were counted on on Columbia blood agar (CFU were counted after 24 hours incubation of plates at 37° C.). Data represent fold change CFU/ml count compared to untreated controls (=1). ANOVA with Fisher's LSD test, *P<0.05, ***P<0.001.

Panel B) Biofilms of adherent-invasive Escherichia coli (strain LF82) were formed into the Calgary Biofilm Device (Innovotech, Canada). After 48 hours of incubation (37° C., anaerobic environment), biofilms were exposed to various concentrations of human thrombin (0, 10, 100 to 1000 milliunit/ml). After 24 hours, dispersed bacteria from thrombin-treated biofilms were collected and apically exposed to human epithelial monolayers on 12-wells plates (Caco2, ATCC HB37). Bacterial adhesion to epithelial surface was assessed. Briefly, after 90 minutes of co-culture, cells were vigorously washed in PBS, and lyzed in Triton X100 0.1% for 10 minutes, after which live cell bacteria were counted on on Columbia blood agar (CFU were counted after 24 hours incubation of plates at 37° C.). Data represent fold change CFU/ml count compared to untreated controls (=1). ANOVA with Fisher's LSD test, *P<0.05

Panel C) Mucosa-associated microbiota was extracted from 3 healthy human colon biopsies (collected in Toulouse Hospital under ethic approval CODECOH Colic-DC2015-2443), under anaerobic condition, and were seeded into the Calgary Biofilm Device (Innovotech, Canada) to develop a multispecies biofilm. After 48 hours of incubation (37° C., anaerobic environment), biofilms were exposed to human thrombin (0 and 100 milliunit/ml). After 24 hours, dispersed bacteria from thrombin-treated biofilms were collected and apically exposed to human epithelial monolayers on 12-wells plates (Caco2, ATCC HB37). Bacterial invasion into epithelial cells was assessed. Briefly, after 3 hours of co-culture, cells were incubated in gentamicin (50 μg/ml) for additional 2 hours, then vigorously washed in PBS, and lyzed in Triton X100 0.1% for 10 minutes, after which live cell bacteria were counted on on Columbia blood agar (CFU/ml were counted after 24 hours incubation of plates at 37° C.). Student's t test, *P<0.05.

Results: In this example, the results demonstrated that exposure of human microbiota biofilms to high thrombin concentrations, such as the ones that are detected in the mucosa of IBD patients, increased the virulent properties of these biofilms. Human microbial biofilms exposed to 1 U/ml of thrombin increased their potential to adhere to human intestinal epithelium monolayers (FIG. 7A). Similarly, the E. coli strain LF82 adhered significantly more to human intestinal epithelium monolayers, when previously exposed to thrombin, even at lower concentrations (0.01 U/ml) (FIG. 7B). Finally, the ability of E. coli LF82 to invade human intestinal epithelium monolayers was significantly increased when these E. coli biofilms were previously exposed to Thrombin (0.1 U/ml).

Discussion: Taken together, these results demonstrate that exposure of microbial biofilms to high concentrations of thrombin such as the ones that are detected in the mucosa of IBD patients, induced an increased virulence of these biofilms, detaching bacteria that are more prone to adhere to and to invade human intestinal epithelial cells. This is true for the Adherent/Invasive E. coli strain LF82, but this is also true for a mixed healthy human microbiota, which under the pressure of high concentrations of thrombin, detached pathobionts that adhere to human intestinal epithelial cells. These results illustrate the fact that in humans, high concentrations of thrombin increases the virulence of intestinal biofilms.

### Table Section

TABLE 1
Useful nucleotide sequences for practicing
the invention

| SEQ ID NO | Nucleotide sequences |
|---|---|
| 1) F2 forward | aggcctgacatcaactccac |
| primer | |
| sequence | |
| 2) F2 reverse | ttggaacctcttgagcgagg |
| primer | |
| sequence | |
| 3)GAPDH | caaggtcatccatgacaactttg |
| forward primer | |
| sequence | |
| 4)GAPDH | gggccatccacagtcttctg |
| reverse primer | |
| sequence | |

### REFERENCES

Throughout this application, various references describe the state of the art to which this invention pertains. The disclosures of these references are hereby incorporated by reference into the present disclosure.
1. Ramos G P, Papadakis K A. Mechanisms of disease: Inflammatory bowel diseases. Mayo Clin Proc 2019; 94:155-65.
2. Allaire J M, Crowley S M, Law H T, et al. The intestinal epithelium: Central coordinator of mucosal immunity. Trends Immunol 2018; 39:677-96.
3. Vergnolle N. Protease inhibition as new therapeutic strategy for gi diseases. Gut 2016; 65:1215-24.
4. Cenac N, Andrews C N, Holzhausen M, et al. Role for protease activity in visceral pain in irritable bowel syndrome. J Clin Invest 2007; 117:636-47.
5. Denadai-Souza A, Bonnart C, Tapias N S, et al. Functional proteomic profiling of secreted serine proteases in health and inflammatory bowel disease. Sci Rep 2018; 8:7834.
6. Rolland-Fourcade C, Denadai-Souza A, Cirillo C, et al. Epithelial expression and function of trypsin-3 in irritable bowel syndrome. Gut 2017; 66:1767-78.
7. Motta J P, Magne L, Descamps D, et al. Modifying the protease, antiprotease pattern by elafin overexpression protects mice from colitis. Gastroenterology 2011; 140:1272-82.
8. Roka R, Rosztoczy A, Leveque M, et al. A pilot study of fecal serine-protease activity: A pathophysiologic factor in diarrhea-predominant irritable bowel syndrome. Clin Gastroenterol Hepatol 2007; 5:550-5.
9. Motta J P, Denadai-Souza A, Sagnat D, et al. Active thrombin produced by the intestinal epithelium controls mucosal biofilms. Nat Commun 2019; 10:3224.
10. Scaldaferri F, Lancellotti S, Pizzoferrato M, De Cristofaro R. Haemostatic system in inflammatory bowel diseases: New players in gut inflammation. World J Gastroenterol 2011; 17:594-608.
11. Saibeni S, Saladino V, Chantarangkul V, et al. Increased thrombin generation in inflammatory bowel diseases. Thromb Res 2010; 125:278-82.
12. Takeyama H, Mizushima T, Iijima H, et al. Platelet activation markers are associated with crohn's disease activity in patients with low c-reactive protein. Dig Dis Sci 2015; 60:3418-23.
13. Owczarek D, Undas A, Foley J H, et al. Activated thrombin activatable fibrinolysis inhibitor (tafia) is associated with inflammatory markers in inflammatory bowel diseases tafia level in patients with ibd. J Crohns Colitis 2012; 6:13-20.
14. Zhang J, Guo Z, Yang W, et al. D-dimer levels are correlated with disease activity in crohn's patients. Oncotarget 2017; 8:63971-7.
15. Souto J C, Martinez E, Roca M, et al. Prothrombotic state and signs of endothelial lesion in plasma of patients with inflammatory bowel disease. Dig Dis Sci 1995; 40:1883-9.
16. Kohoutova D, Pecka M, Cihak M, et al. Prevalence of hypercoagulable disorders in inflammatory bowel disease. Scand J Gastroenterol 2014; 49:287-94.
17. Stadnicki A. Involvement of coagulation and hemostasis in inflammatory bowel diseases. Curr Vasc Pharmacol 2012; 10:659-69.
18. Prajapati D N, Newcomer J R, Emmons J, Abu-Hajir M, Binion D G. Successful treatment of an acute flare of steroid-resistant crohn's colitis during pregnancy with unfractionated heparin. Inflamm Bowel Dis 2002; 8:192-5.
19. Ang Y S, Mahmud N, White B, et al. Randomized comparison of unfractionated heparin with corticosteroids in severe active inflammatory bowel disease. Aliment Pharmacol Ther 2000; 14:1015-22.
20. Vergnolle N, Comera C, More J, Alvinerie M, Bueno L. Expression and secretion of lipocortin 1 in gut inflammation are not regulated by pituitary-adrenal axis. Am J Physiol 1997; 273:R623-9.
21. Vergnolle N, Comera C, Bueno L. Annexin 1 is overexpressed and specifically secreted during experimentally induced colitis in rats. Eur J Biochem 1995; 232:603-10.
22. Hyun E, Andrade-Gordon P, Steinhoff M, Vergnolle N. Protease-activated receptor-2 activation: A major actor in intestinal inflammation. Gut 2008; 57:1222-9.
23. Woting A, Blaut M. Small intestinal permeability and gut-transit time determined with low and high molecular weight fluorescein isothiocyanate-dextrans in c3h mice. Nutrients 2018; 10.
24. Chin A C, Lee W Y, Nusrat A, Vergnolle N, Parkos C A. Neutrophil-mediated activation of epithelial protease-activated receptors-1 and -2 regulates barrier function and transepithelial migration. J Immunol 2008; 181:5702-10.
25. Chin A C, Vergnolle N, MacNaughton W K, et al. Proteinase-activated receptor 1 activation induces epithelial apoptosis and increases intestinal permeability. Proc Natl Acad Sci USA 2003; 100:11104-9.
26. Thakur R, Chattopadhyay P, Ghosh S S, Mukherjee A K. Elucidation of procoagulant mechanism and pathophysiological significance of a new prothrombin activating metalloprotease purified from daboia russelii russelii venom. Toxicon 2015; 100:1-12.
27. Stojanovski B M, Pelc L A, Zuo X, Pozzi N, Cera E D. Enhancing the anticoagulant profile of meizothrombin. Biomol Concepts 2018; 9:169-75.
28. Soslau G, Goldenberg S J, Class R, Jameson B. Differential activation and inhibition of human platelet thrombin receptors by structurally distinct alpha-, beta- and gamma-thrombin. Platelets 2004; 15:155-66.
29. Bradford H N, Krishnaswamy S. Meizothrombin is an unexpectedly zymogen-like variant of thrombin. J Biol Chem 2012; 287:30414-25.
30. Vergnolle N, Derian C K, D'Andrea M R, Steinhoff M, Andrade-Gordon P. Characterization of thrombin-induced leukocyte rolling and adherence: A potential proinflammatory role for proteinase-activated receptor-4. J Immunol 2002; 169:1467-73.
31. Houle S, Papez M D, Ferazzini M, Hollenberg M D, Vergnolle N. Neutrophils and the kallikrein-kinin system in proteinase-activated receptor 4-mediated inflammation in rodents. Br J Pharmacol 2005; 146:670-8.
32. Sebert M, Denadai-Souza A, Quaranta M, et al. Thrombin modifies growth, proliferation and apoptosis of human colon organoids: A protease-activated receptor 1- and protease-activated receptor 4-dependent mechanism. Br J Pharmacol 2018; 175:3656-68.
33. Motta J P, Bermudez-Humaran L G, Deraison C, et al. Food-grade bacteria expressing elafin protect against inflammation and restore colon homeostasis. Sci Transl Med 2012; 4:158ra44.
34. Oikonomopoulou K, Hansen K K, Saifeddine M, et al. Kallikrein-mediated cell signalling: Targeting proteinase-activated receptors (pars). Biol Chem 2006; 387:817-24.
35. Oikonomopoulou K, Hansen K K, Saifeddine M, et al. Proteinase-mediated cell signalling: Targeting proteinase-activated receptors (pars) by kallikreins and more. Biol Chem 2006; 387:677-85.
36. Renesto P, Si-Tahar M, Moniatte M, et al. Specific inhibition of thrombin-induced cell activation by the neutrophil proteinases elastase, cathepsin g, and proteinase 3: Evidence for distinct cleavage sites within the aminoterminal domain of the thrombin receptor. Blood 1997; 89:1944-53.
37. Dulon S, Leduc D, Cottrell G S, et al. Pseudomonas aeruginosa elastase disables proteinase-activated receptor 2 in respiratory epithelial cells. Am J Respir Cell Mol Biol 2005; 32:411-9.
38. Nguyen G C, Bernstein C N, Bitton A, et al. Consensus statements on the risk, prevention, and treatment of venous thromboembolism in inflammatory bowel disease: Canadian association of gastroenterology. Gastroenterology 2014; 146:835-48 e6.
39. Baker N C, Lipinski M J, Lhermusier T, Waksman R. Overview of the 2014 food and drug administration cardiovascular and renal drugs advisory committee meeting about vorapaxar. Circulation 2014; 130:1287-94.

1. A method of treating an inflammatory bowel disease (IBD) in a patient in need thereof, comprising
administering to the patient a therapeutically effective amount of a direct thrombin antagonist, wherein said direct thrombin antagonist is administered locally in the gut of the patient to, and wherein said direct thrombin antagonist directly binds to a thrombin protein or a thrombin encoding nucleic acid sequence and inhibits mucosal inflammation through blocking PAR-1 (Protease-Activated Receptor-1) activity and/or decreasing virulent properties of human mucosa-associated biofilms.

2. The method according to claim 1 wherein the inflammatory bowel disease (IBD) is ulcerative colitis (UC) or Crohn's disease (CD).

3. The method according to claim 1 wherein said antagonist is
1) an inhibitor of thrombin activity and/or
2) an inhibitor of thrombin gene expression.

4. The method according to claim 3 wherein said inhibitor of thrombin activity is selected from the group consisting of a small organic molecule; an anti-thrombin neutralizing antibody, a polypeptide, an aptamer and a polysaccharide.

5. The method according to claim 4, wherein the polypeptide is a serpin family polypeptide selected from the group consisting of antithrombin, heparin cofactor II, protein C inhibitor and protease Nexin 1.

6. The method according to claim 5, wherein the serpin family polypeptide is delivered directly to the gut by administering to the patient a food-grade bacterium comprising a gene encoding the serpin family polypeptide.

7. The method according to claim 3 wherein the inhibitor of thrombin gene expression is selected from the group consisting of an antisense oligonucleotide, a nuclease, siRNA, shRNA or a ribozyme nucleic acid sequence.

8. The method according to claim 1, wherein a high level of thrombin is present in a biological sample from the patient.

9. A method of treating a patient afflicted with an inflammatory bowel disease (IBD) comprising,
administering to the patient a therapeutically effective amount of a recombinant food-grade bacterium comprising a gene coding for a member of the serpin family of proteins or an active fragment of a member of the serpin family of proteins, wherein said recombinant food-grade bacterium is administered locally to the gut of the patient.

10. The method of claim 1, wherein the thrombin encoding nucleic acid sequence is DNA or mRNA.

11. The method of claim 9, wherein the member of the serpin family of proteins is antithrombin, heparin cofactor II, a protein C inhibitor or protease Nexin 1.
